(19) United States
(12) Patent Application Publication
(10) Pub. No.: US 2023/0406943 A1
(43) Pub. Date: Dec. 21, 2023

(54) METHODS FOR TREATING INFLAMMATORY SKIN CONDITIONS

(71) Applicant: CSL INNOVATION PTY LTD, Melbourne VIC (AU)

(72) Inventors: Catherine Mary OWCZAREK, Melbourne VIC (AU); Angel Francisco LOPEZ, Medindie (AU); Ho Kwok YIP, Broadview (AU); Damon John TUMES, Flinders Park (AU)

(21) Appl. No.: 18/037,219

(22) PCT Filed: Dec. 3, 2021
(86) PCT No.: PCT/AU2021/051442

§ 371 Date: May 16, 2023

(30) Foreign Application Priority Data
Dec. 4, 2020 (AU) 2020904494
Jun. 17, 2021 (AU) 2021901818

(51) Int. Cl. C07K 16/28; A61P 17/00

(52) U.S. Cl. CPC C07K 16/2866; A61P 17/00; A61K 2039/505

(57) ABSTRACT

The present disclosure relates to methods of treating inflammatory skin conditions using compounds that bind to CD131 and inhibit GM-CSF and IL-5 signaling. The present disclosure also relates to compounds for use in the treatment or prevention of an inflammatory skin condition, as well as the use of such compounds in the manufacture of medicaments for the treatment or prevention of inflammatory skin conditions.

### RELATED APPLICATION DATA

The present application claims priority from Australian Patent Application No. 2020904494 filed on 4 Dec. 2020 entitled “Methods for treating inflammatory skin conditions” and Australian Patent Application No. 2021901818 filed on 17 Jun. 2021 entitled “Methods for treating inflammatory skin conditions”. The entire contents of both applications are hereby incorporated by reference.

### SEQUENCE LISTING

The present application is filed together with a Sequence Listing in electronic form. The entire contents of the Sequence Listing are hereby incorporated by reference.

### FIELD

The present disclosure relates to methods of treating inflammatory skin conditions in a subject.

### BACKGROUND

Inflammatory skin conditions such as atopic dermatitis (AD) and allergic contact dermatitis (ACD) affect large proportions of the general population. For example, AD affects 10-15% of children and ACD affects 15-20% of the general population worldwide. Many patients carry their inflammatory skin condition into adulthood, hence for some, the disease is lifelong. Therefore, the development of an effective treatment for such conditions would represent a significant public health advance.

The underlying causes of inflammatory skin conditions include a combination of genetic and environmental factors, and several overlapping immunological pathways are thought to contribute to the development of disease. Because of the multifactorial nature of inflammatory skin conditions, the efficacy of currently available drugs is limited. The mainstay of treatment has been topical therapy, which includes emollients, corticosteroids, and calcineurin inhibitors in mild disease. However, these treatments are not sufficient in recalcitrant inflammation in patients with moderate to severe disease and have been known to lead to substantial adverse side effects. Despite poor evidence, systemic corticosteroids in combination with antihistamines or the use of time-consuming phototherapy are sometimes used. Patients with severe disease refractory to conventional therapy may be treated with systemic non-steroidal treatment options with varying levels of evidence including cyclosporine A, methotrexate, azathioprine, and mycophenolate mofetil. Such drugs are non-specific immunosuppressants, each with their own unique set of undesirable side effects.

Therefore, there is a need for new interventions for treating inflammatory skin conditions.

### SUMMARY

In producing the present invention, the inventors developed a transgenic mouse model of ACD. The transgenic mouse expresses human CD131 (0c) and is useful for investigating the effects of targeting human CD131. The inventors found that inhibition of granulocyte-macrophage colony stimulating factor (GM-CSF) and interleukin (IL) 5 signaling, using an antibody that binds to CD131, reduced inflammatory cell accumulation and ameliorates disease. These findings provide the basis for methods of treating inflammatory skin conditions, such as ACD.

Accordingly, in an example, the present disclosure provides a method for treating an inflammatory skin condition in a subject, the method comprising administering one or more compound(s) that neutralize signaling by GM-CSF and IL-5 to the subject. Similarly, the present disclosure provides one or more compound(s) that neutralize signaling by GM-CSF and IL-5 for use in treating an inflammatory skin condition in a subject. The present disclosure also provides use of one or more compound(s) that neutralize signaling by GM-CSF and IL-5 in the manufacture of a medicament for treating an inflammatory skin condition.

In another example, the present disclosure provides a method for treating an inflammatory skin condition in a subject, the method comprising administering a compound that binds to CD131 and neutralizes signaling by GM-CSF and IL-5 to the subject.

The present disclosure also provides a compound that binds to CD131 and neutralizes signaling by GM-CSF and IL-5 for use in treating an inflammatory skin condition in a subject. The present disclosure also provides use of a compound that binds to CD131 and neutralizes signaling by GM-CSF and IL-5 in the manufacture of a medicament for treating an inflammatory skin condition.

The methods of the present disclosure are suitable for treating any type of inflammatory skin condition, particularly those associated with GM-CSF and/or IL-5 signaling. In some examples, the inflammatory skin condition is a hypersensitivity, autoimmune condition, allergic condition, neutrophilic dermatosis, atopic condition, autoinflammatory condition and/or T cell mediated condition.

In one example, the inflammatory skin condition is ACD, AD, chronic spontaneous urticaria, prurigo nodularis, psoriasis, psoriasis guttata, inverse psoriasis, pustular psoriasis, plaque psoriasis, psoriatic erythroderma, amicrobial pustulosis of the folds (APF), CARD14-mediated pustular psoriasis (CAMPS), cryopyrin associated periodic syndromes (CAPS), deficiency of interleukin-1 receptor (DIRA), deficiency of interleukin-36 receptor antagonist (DIRTA), hidradenitis suppurativa (HS), palmoplantar pustulosis (PPP), pyogenic arthritis, pyoderma gangrenosum and acne (PAPA), pyoderma gangrenosum (PG), Still's disease, Sweet syndrome, subcorneal pustulosis (Sneddon-Wilkinson), acute generalized exanthematic pustulosis, infantile acropustulosis, synovitis, pustulosis, hyperostosis and osteitis (SAPHO) syndrome, bowel-associated dermatosis-arthritis syndrome (BADAS), neutrophilic dermatosis of the dorsal hands, erythema elevatum diutinum, Pyoderma gangrenosumacute febrile neutrophilic dermatosis, xerotic eczema, dyshidrotic eczema, vesicular palmar eczema, acne vulgaris, contact dermatitis, melisma, dermatomyositis, exfoliative dermatitis, hand eczema, pompholyx, bullous pemphigoid, pemphigusrosacea, rosacea, rosacea due to sarcoidosis, rosacea due to scleroderma, rosacea due to Sweet syndrome, rosacea due to systemic lupus erythematosus, rosacea due to urticaria, rosacea due to herpetic pain, Sweet's disease, neutrophilic hydrodenitis, sterile pustule, drug rash, urticarial, seborrheic dermatitis, Pityriasis rosea, Kikuchi's disease of the skin, pruritic urticarial papules and plaques of pregnancy, Stevens-Johnson syndrome and toxic epidermal necrolysis, tattoo reaction, Wells syndrome (eosinophilic cellulitis), reactive arthritis (Reiter syndrome), bowel-associated dermatosis-arthritis syndrome, rheumatoid neutrophilic dermatosis, neutrophilic eccrine hidradenitis, neutrophilic skin disease of dorsum of hand, balanitis circumscripta plasmacellularis, balanoposthitis, Behcet's disease, erythema annulare centrifugum, erythema dyschromicum perstans, erythema multiforme, granuloma annulare, dermatitis of hand, lichen nitidus, lichen planus, lichen sclerosus et atrophicus, lichen simplex chronicus, lichen spinulosus, nummular dermatitis, sarcoidosis, subkeratinous pustular dermatosis, urticaria, transient acantholytic dermatosis, and urushiol-induced contact dermatitis.

In one example, the inflammatory skin condition is a hypersensitivity.

In some examples, the hypersensitivity is a type I hypersensitivity. In some examples, the type I hypersensitivity is associated with angioedema, urticaria, a bee sting reaction, or a latex allergy.

In some examples, the hypersensitivity is a type II hypersensitivity. In some examples, the type II hypersensitivity is bullous pemphigoid or pemphigus vulgaris.

In some examples, the hypersensitivity is a type III hypersensitivity. In some examples, the type III hypersensitivity is Henoch-Schdnlein purpura, small-vessel vasculitis, or systemic lupus erythematosus.

In some examples, the hypersensitivity is a type IV hypersensitivity.

In some examples, the type IV hypersensitivity is allergic contact dermatitis, a morbilliform drug reaction, drug hypersensitivity syndrome (formerly known as drug reaction with eosinophilia and systemic symptoms [DRESS]), erythema multiforme, lichenoid drug eruptions, Steven-Johnson syndrome (SJS) or toxic epidermal necrolysis (TEN).

In some examples, the inflammatory skin condition is a contact dermatitis.

In some examples, the contact dermatitis is irritant contact dermatitis (ICD).

In some examples, the contact dermatitis is allergic contact dermatitis (ACD). As described in the Examples, the inventors found that, in a mouse model of ACD, inhibition of GM-CSF and IL-5 signaling, by an anti-CD131 antibody, reduced inflammatory cell accumulation and ameliorated disease. Thus, the methods of the present disclosure are particularly well suited for treating ACD and other cell-mediated inflammatory conditions.

In some examples, the inflammatory skin condition is a cell-mediated condition. In one example, the inflammatory skin condition is a T cell-mediated condition. In some examples, the inflammatory skin condition is associated with mast cell and/or neutrophil infiltration at the site of inflammation.

In some examples, the inflammatory skin condition is an antibody-mediated condition. In some examples, the inflammatory skin condition is an IgE-mediated condition and/or a condition associated with elevated serum IgE levels (e.g., atopic dermatitis). In other examples, the inflammatory skin condition is not an IgE-mediated condition and/or is not a condition associated with elevated serum IgE levels.

In some examples, the inflammatory skin condition is atopic dermatitis.

In some examples, the inflammatory skin condition is an autoimmune condition. In some examples, the autoimmune condition is psoriasis, systemic sclerosis, dermatomyositis, vitiligo, alopecia areata, lichen sclerosus. In one example, the autoimmune condition is an autoimmune blistering disease. In some examples, autoimmune blistering disease is pemphigus vulgaris, pemphigus foliaceus, paraneoplastic pemphigus, bullous pemphigoid, mucous membrane pemphigoid, pemphigoid gestationis, dermatitis herpetiformis, linear IgA bullous dermatosis, epidermolysis bullosa acquisita, or bullous systemic lupus erythematosus.

In some examples, the inflammatory skin condition is an autoinflammatory skin disease. In some examples, the autoinflammatory skin disease is familial mediterranean fever (FMF), tumour necrosis factor receptor-associated periodic fever syndrome (TRAPS), hyper-IgD syndrome (HIDS), cryopyrin-associated periodic syndromes (CAPS), familial cold autoinflammatory syndrome (FCAS), Muckle-Wells syndrome (MWS), neonatal onset multisystem inflammatory disease/chronic Infantile neurologic cutaneous arthropathy syndrome (NOMID/CINCA), syndrome of pyogenic arthritis, pyoderma gangrenosum and acne (PAPA syndrome, PAPAS, PAPGA syndrome), juvenile systemic granulomatosis (Blau syndrome, early onset sarcoidosis), deficiency of interleukin-1 receptor antagonist (DIRA), mevalonic aciduria, Majeed syndrome, Schnitzler syndrome, Behcet disease, hidradenitis suppurativa, or syndrome of periodic fever, aphthous stomatitis, pharyngitis and adenitis (PAPAS, PFAPA syndrome).

In some examples, the inflammatory skin condition is a neutrophilic dermatosis. In one example, the neutrophilic dermatosis is amicrobial pustulosis of the folds (APF); plaque psoriasis; CARD14-mediated pustular psoriasis (CAMPS); cryopyrin associated periodic syndromes (CAPS); deficiency of interleukin-1 receptor (DIRA); deficiency of interleukin-36 receptor antagonist (DIRTA); hidradenitis suppurativa (HS); palmoplantar pustulosis; pyogenic arthritis; pyoderma gangrenosum and acne (PAPA); pyoderma gangrenosum, acne, and hidradenitis suppurativa (PASH); pyoderma gangrenosum (PG); skin lesions of Behcet's disease; Still's disease; Sweet syndrome; subcorneal pustulosis (Sneddon-Wilkinson); pustular psoriasis; palmoplantar pustulosis; acute generalized exanthematic pustulosis; infantile acropustulosis; synovitis, acne, pustulosis; hyperostosis and osteitis (SAPHO) syndrome; bowel-associated dermatosis-arthritis syndrome (BADAS); neutrophilic dermatosis of the dorsal hands; neutrophilic eccrine hidradenitis; erythema elevatum diutinum; or Pyoderma gangrenosum. In one example, the neutrophilic dermatosis is hidradenitis suppurativa (HS) or palmoplantar pustulosis (PPP).

The present inventors found that in their mouse model of ACD, administration of an antibody that binds to CD131, and neutralizes signaling by GM-CSF and IL-5, reduced swelling, mast cell infiltration, eosinophil infiltration, CD8+ T cell infiltration and neutrophil infiltration, relative to mice administered an isotype control antibody. Thus, in some examples, administration of the compound that binds to CD131 and neutralizes signaling by GM-CSF and IL-5:
a) reduces swelling at the site of inflammation, and/or
b) reduces mast cell infiltration at the site of inflammation, and/or
c) reduces neutrophil infiltration at the site of inflammation.

In some examples, administration of the compound that binds to CD131 and neutralizes signaling by GM-CSF and IL-5:
a) reduces swelling at the site of inflammation, and/or
b) reduces mast cell infiltration at the site of inflammation, and/or
c) reduces neutrophil infiltration at the site of inflammation, and/or
d) reduces CD8+ T cell infiltration at the site of inflammation, and/or
e) reduces eosinophil infiltration at the site of inflammation.

In some examples, administration of the compound that binds to CD131 and neutralizes signaling by GM-CSF and IL-5:
a) reduces CD8+ T cell infiltration at the site of inflammation, and/or
b) reduces eosinophil infiltration at the site of inflammation.

Suitable methods for assessing the above are described herein and will be known by those skilled in the art.

In some examples, the compound that binds to CD131 and neutralizes signaling by GM-CSF and IL-5 also neutralizes signaling by IL-3.

In one example, the compound that binds to CD131 and neutralizes signaling by GM-CSF and IL-5 inhibits GM-CSF-induced proliferation of TF-1 cells with an IC₅₀ of at least 600 nM or 500 nM. For example, the IC₅₀ is at least about 400 nM. For example, the IC₅₀ is at least about 300 nM or 200 nM or 100 nM. For example, the IC₅₀ is at least about 50 nM. For example, the IC₅₀ is at least about 10 nM or 5 nM or 1 nM. In one example, the IC₅₀ is at least about 1 nM. For example, the IC₅₀ is at least about 0.9 nM or 0.8 nM or 0.6 nM. In one example, the IC₅₀ is at least about 0.5 nM. In one example, the IC₅₀ is at least about 0.4 nM. In one example, the IC₅₀ is at least about 0.3 nM.

In one example, the compound that binds to CD131 and neutralizes signaling by GM-CSF and IL-5 inhibits IL-5-induced proliferation of TF-1 cells with an IC₅₀ of at least 600 nM or 500 nM. For example, the IC₅₀ is at least about 400 nM. For example, the IC₅₀ is at least about 300 nM or 200 nM or 100 nM. For example, the IC₅₀ is at least about 50 nM. For example, the IC₅₀ is at least about 10 nM or 5 nM or 1 nM. In one example, the IC₅₀ is at least about 5 nM. For example, the IC₅₀ is at least about 4 nM. In one example, the IC₅₀ is at least about 4.5 nM or at least about 4.6 nM or at least about 4.7 nM. In one example, the IC₅₀ is at least about 4.6 nM.

In one example, the compound that binds to CD131 and neutralizes signaling by GM-CSF and IL-5 inhibits IL-3-induced proliferation of TF-1 cells with an IC₅₀ of at least 600 nM or 500 nM. For example, the IC₅₀ is at least about 400 nM. For example, the IC₅₀ is at least about 300 nM or 200 nM or 100 nM. For example, the IC₅₀ is at least about 50 nM. For example, the IC₅₀ is at least about 10 nM or 5 nM or 1 nM. In one example, the IC₅₀ is at least about 1 nM. For example, the IC₅₀ is at least about 0.9 nM or 0.8 nM or 0.6 nM. In one example, the IC₅₀ is at least about 0.5 nM. In one example, the IC₅₀ is at least about 0.2 nM or at least about 0.1 nM. In one example, the IC₅₀ is at least about 0.15 nM.

Methods for determining the IC₅₀ are described herein and include culturing TF-1 cells (e.g., about 1×10⁴ TF-1 cells) in the presence of the compound that binds to CD131 (e.g., for at least about 3 minutes or 1 hour, such as about 30 minutes) prior to adding the relevant growth factor (GM-CSF, IL-3 and/or IL-5) and culturing the cells further (e.g., for at least about 48 hours or at least about 72 hours or at least about 96 hours, e.g., for about 72 hours) and then determining cell proliferation. Cell proliferation can be determined by growing the cells in the presence of 3[H]-thymidine for about 6 hours and determining ³[H]-thymidine incorporation, e.g., by liquid-scintillation counting. By determining proliferation in a variety of concentrations of the compound that binds to CD131 an IC₅₀ can be determined.

In some examples, the compound that binds to CD131 and neutralizes signaling by GM-CSF and IL-5 inhibits
a) GM-CSF-induced proliferation of TF-1 cells with an IC₅₀ of at least 100 nM; and/or
b) IL-5-induced proliferation of TF-1 cells with an IC₅₀ of at least 100 nM; and/or
c) IL-3-induced proliferation of TF-1 cells with an IC₅₀ of at least 100 nM.

In some examples, the compound that binds to CD131 and neutralizes signaling by GM-CSF and IL-5 inhibits
a) GM-CSF-induced proliferation of TF-1 cells with an IC₅₀ of at least 50 nM; and/or
b) IL-5-induced proliferation of TF-1 cells with an IC₅₀ of at least 50 nM; and/or
c) IL-3-induced proliferation of TF-1 cells with an IC₅₀ of at least 50 nM.

In some examples, the compound that binds to CD131 and neutralizes signaling by GM-CSF and IL-5 inhibits
a) GM-CSF-induced proliferation of TF-1 cells with an IC₅₀ of at least 10 nM; and/or
b) IL-5-induced proliferation of TF-1 cells with an IC₅₀ of at least 10 nM; and/or
c) IL-3-induced proliferation of TF-1 cells with an IC₅₀ of at least 10 nM.

In one example, the compound that binds to CD131 reduces or prevents IL-3 and/or GM-CSF-induced STAT-5 signaling.

In one example, the compound that binds to CD131 reduces or prevents IL-3-induced STAT-5 signaling with an IC₅₀ of about 20 nM or less. In one example, the pStat-5 IC₅₀ IL-3 is about 10 nM or less, or about 9 nM or less, or about 8 nM or less. In one example, the pStat-5 IC₅₀ IL-3 is about 7.5 nM or less, for example 7.3 nM.

In one example, the compound that binds to CD131 reduces or prevents GM-CSF-induced STAT-5 signaling with an IC₅₀ of about 60 nM or less. In one example, the pStat-5 IC₅₀ GM-CSF is about 50 nM or less, or about 45 nM or less or about 40 nM or less. In one example, the compound that binds to CD131 reduces or prevents GM-CSF-induced STAT-5 signaling with an IC₅₀ of about 40 nM.

For example, the compound can be contacted to a cell (e.g., a TF-1 cell) comprising a beta-lactamase reporter gene under control of the interferon regulatory factor 1 (irf1) response element in the presence of IL-3 and/or GM-CSF. Cells are also contacted with a suitable substrate (e.g., a negatively charged fluorescent beta-lactamase substrate, such as CCF2 or CCF4) and the change in signal (e.g., fluorescence) determined. A reduced change in signal in a positive control (i.e., cells contacted with IL-3 and/or GM-CSF in the absence of the protein or antibody) indicates that the compound reduces or prevents IL-3 and/or GM-CSF-induced STAT-5 signaling.

In one example, the compound that binds to CD131 has one or more of the following activities:
(i) reduces or inhibits activation of isolated human neutrophils by GM-CSF as determined by reducing or inhibiting GM-CSF-induced increase in neutrophil cell size;
(ii) reduces or inhibits IL-3-induced IL-8 secretion by human basophils;
(iii) reduces or prevents IL-3-mediated survival or plasmacytoid dendritic cells (pDCs);
(iv) reduces or prevents activation of human peripheral blood eosinophils by IL-5 as determined by assessing change in forward scatter assessed by flow cytometry;
(v) reduces or prevents survival of human peripheral blood eosinophils in the presence of IL-5 and/or GM-CSF and/or IL-3;
(vi) reduces or prevents IL-3-induced tumor necrosis factor (TNF) α release from human mast cells;
(vii) reduces or prevents IL-3-induced IL-13 release from human mast cells;
(viii) reduces or prevents potentiation of IgE-mediated IL-8 release from human mast cells by IL-3 and/or IL-5 and/or GM-CSF;
(ix) reduces or prevents formation of colony forming units-granulocytes-macrophages (CFU-GM) by CD34+ human bone marrow cells cultured in the presence of stem cell factor (SCF), GM-CSF, IL-3 and IL-5;
(x) reduces the size or weight of polyps in a mouse xenograft model of human nasal polyposis; and/or
(xi) reduces the number of B cells in a polyp in a mouse xenograft model of human nasal polyposis.

In one example, the compound that binds to CD131 does not substantially or significantly inhibit proliferation of TF-1 cells in response to one or more of erythropoietin, IL-6, IL-4 or stem cell factor. Methods for determining the ability of the compound that binds to CD131 to inhibit proliferation of TF-1 cells in respect to a cytokine or growth factor are described herein and are readily adaptable to the present example of the disclosure.

In some examples, the compound that binds to CD131 and neutralizes signaling by GM-CSF and IL-5 is a protein comprising an antigen binding site that binds to CD131. In some examples, the antigen binding site is an antigen binding site of an antibody or a single domain antibody. In some examples, the antigen binding site comprises one or more CDRs.

Reference herein to a compound or protein or antibody that “binds to” CD131 provides literal support for a compound or protein or antibody that “binds specifically to” or “specifically binds to” CD131.

In one example, the K_{D} of the protein for a polypeptide comprising a sequence set forth in SEQ ID NO: 5 is about 10 nM or less, when the polypeptide is immobilized on a solid surface and the K_{D} is determined by surface plasmon resonance.

In one example, the K_{D} is 10 nM or less, for example, 5 nM or less or 4 nM or less, or 3 nM or less or 2 nM or less. In one example, the K_{D} is 1 nM or less. In one example, the K_{D} is 0.9 nM or less or 0.7 nM or less or 0.8 nM or less or 0.7 nM or less or 0.6 nM or less. In one example, the K_{D} is 0.5 nM or less. In one example, the K_{D} is 0.4 nM or less. In one example, the K_{D} is 0.3 nM or less. In one example, the K_{D} is 0.2 nM or less.

In one example, the protein comprising an antigen binding site binds to a cell expressing CD131 (e.g., a neutrophil or an eosinophil or a TF-1 cell) with a K_{D} of about 10 nM or less, e.g., using a competition assay using labeled and unlabeled protein or antibody. In one example, the K_{D} is 5 nM or less or 4 nM or less, or 3 nM or less or 2 nM or less. In one example, the K_{D} is 1 nM or less. In one example, the K_{D} is 0.9 nM or less or 0.7 nM or less or 0.8 nM or less or 0.7 nM or less or 0.6 nM or less.

In one example, the K_{D} is about 300 nM or less for a neutrophil.

In one example, the K_{D} is about 700 nM or less for an eosinophil.

In one example, the K_{D} is about 400 nM or less for a TF-1 cell.

In one example, the protein comprising an antigen binding site is a protein comprising one or more antibody variable regions. In one example, the protein comprises a heavy chain variable region (V_{H}). In one example, the protein comprises a light chain variable region (V_{L}). In one example, the protein comprises a V_{H} and a V_{L}. In some examples, the V_{H} and a V_{L} are in the same polypeptide chain. In other examples, the V_{H} and a V_{L} are in separate polypeptide chains.

In some examples, the protein is a single domain antibody (sdAb).

In some examples, the protein comprises a Fv.

In some examples, the protein comprises:
(i) a single chain Fv fragment (scFv);
(ii) a dimeric scFv (di-scFv); or
(iii) a diabody;
(iv) a triabody;
(v) a tetrabody;
(vi) a Fab;
(vii) a F(ab′)₂;
(viii) a Fv;
(ix) one of (i) to (viii) linked to a constant region of an antibody, Fc or a heavy chain constant domain (C_{H}) 2 and/or C_{H}3;
(x) one of (i) to (viii) linked to albumin or a functional fragment or variants thereof or a protein that binds to albumin; or
(xi) an antibody.

In some examples, the protein is selected from the group consisting of:
(i) a single chain Fv fragment (scFv);
(ii) a dimeric scFv (di-scFv); or
(iii) a diabody;
(iv) a triabody;
(v) a tetrabody;
(vi) a Fab;
(vii) a F(ab′)₂;
(viii) a Fv;
(ix) one of (i) to (viii) linked to a constant region of an antibody, Fc or a heavy chain constant domain (C_{H}) 2 and/or C_{H}3;
(x) one of (i) to (viii) linked to albumin, functional fragments or variants thereof or a protein (e.g., antibody or antigen binding fragment thereof) that binds to albumin; or
(xi) an antibody.

In one example, the protein comprises an Fc region.

In one example, the protein comprises one or more amino acid substitutions that increase the half-life of the protein. In one example, the antibody comprises a Fc region comprising one or more amino acid substitutions that increase the affinity of the Fc region for the neonatal Fc receptor (FcRn).

In one example, the protein is an antibody, for example, a monoclonal antibody.

In one example, the antibody is a naked antibody.

In one example, the protein (or antibody) is chimeric, de-immunized, humanized, human or primatized.

In one example, the protein or antibody is human.

Exemplary antibodies include 9A2-VR24.29 (also, referred to as “CSL311”) described in WO 2017/088028 and BION-1 described in Sun et al. (1999) Blood 94:1943-1951.

In one example, the protein comprises a human constant region, e.g., an IgG constant region, such as an IgG1, IgG2, IgG3 or IgG4 constant region or mixtures thereof. In the case of a protein comprising a V_{H} and a V_{L}, the V_{H} can be linked to a heavy chain constant region and the V_{L} can be linked to a light chain constant region.

The C-terminal lysine of the heavy chain constant region of a whole antibody (or a protein comprising a constant region or a C_{H}3) may be removed, for example, during production or purification of the protein or antibody, or by recombinantly engineering the nucleic acid encoding the heavy chain. Accordingly, whole antibodies (or CD131-binding compounds) may comprise populations with all C-terminal lysine residues removed, populations with no C-terminal lysine residues removed, and/or populations having a mixture of protein with and without the C-terminal lysine residue. In some examples, the populations may additionally comprise protein in which the C-terminal lysine residue is removed in one of the heavy chain constant regions. Similarly, a composition of whole antibodies may comprise the same or a similar mix of antibody populations with or without the C-terminal lysine residue.

In one example, the protein is within a composition. For example, the composition comprises a protein comprising an antigen binding site or an antibody as described herein. In one example, the composition additionally comprises one or more variants of the protein or antibody. For example, that comprises a variant missing an encoded C-terminal lysine residue, a deamidated variant and/or a glycosylated variant and/or a variant comprising a pyroglutamate, e.g., at the N-terminus of a protein and/or a variant lacking a N-terminal residue, e.g., a N-terminal glutamine in an antibody or V region and/or a variant comprising all or part of a secretion signal. Deamidated variants of encoded asparagine residues may result in isoaspartic, and aspartic acid isoforms being generated or even a succinamide involving an adjacent amino acid residue. Deamidated variants of encoded glutamine residues may result in glutamic acid. Compositions comprising a heterogeneous mixture of such sequences and variants are intended to be included when reference is made to a particular amino acid sequence.

In one example, a protein or antibody as described herein comprises a constant region of an IgG4 antibody or a stabilized constant region of an IgG4 antibody. In one example, the protein or antibody comprises an IgG4 constant region with a proline at position 241 (according to the numbering system of Kabat (Kabat et al., Sequences of Proteins of Immunological Interest Washington DC United States Department of Health and Human Services, 1987 and/or 1991)).

In one example, the heavy chain constant region comprises a sequence set forth in SEQ ID NO: 16. In one example, the protein, or a composition comprising the protein, comprises a heavy chain constant region, including a stabilized heavy chain constant region, comprising a mixture of sequences fully or partially with or without the C-terminal lysine residue.

In some examples, the protein comprises an antibody variable region that binds to CD131 and competitively inhibits the binding of antibody 9A2-VR24.29 comprising a V_{H} comprising a sequence set forth in SEQ ID NO: 6 and a V_{L} comprising a sequence set forth in SEQ ID NO: 7 to CD131.

In some examples, the protein comprises an antibody variable region that binds to CD131 and competitively inhibits the binding of antibody 9A2-VR24.29 comprising a V_{H} comprising a sequence set forth in SEQ ID NO: 6 and a light chain comprising a sequence set forth in SEQ ID NO: 7 to CD131.

In some examples, the protein comprises an antibody variable region that binds to CD131 and competitively inhibits the binding of antibody 9A2-VR24.29 comprising a V_{H} comprising a sequence set forth in SEQ ID NO: 6 and a V_{L} comprising a sequence set forth in SEQ ID NO: 18 to CD131.

In some examples, the protein binds to the same or an overlapping epitope in CD131 as antibody 9A2-VR24.29 comprising a V_{H} comprising a sequence set forth in SEQ ID NO: 6 and a V_{L} comprising a sequence set forth in SEQ ID NO: 7 to CD131.

In some examples, the protein binds to the same or an overlapping epitope in CD131 as antibody 9A2-VR24.29 comprising a V_{H} comprising a sequence set forth in SEQ ID NO: 6 and a light chain comprising a sequence set forth in SEQ ID NO: 7 to CD131.

In some examples, the protein binds to the same or an overlapping epitope in CD131 as antibody 9A2-VR24.29 comprising a V_{H} comprising a sequence set forth in SEQ ID NO: 6 and a V_{L} comprising a sequence set forth in SEQ ID NO: 18 to CD131.

In some examples, the antigen binding site binds to an epitope within Site 2 of CD131. In this regard, the skilled artisan will be aware that Site 2 of CD131 is made up of residues from two CD131 polypeptides that form a dimer, e.g., Site 2 comprises residues within loops A-B and E-F of domain 1 of one CD131 polypeptide and residues within loops B-C and F-G of another CD131 polypeptide.

In some examples, the antigen binding site binds to an epitope formed upon dimerization of two CD131 polypeptides.

In some examples, the antigen binding site binds to residues within domain 1 of a CD131 polypeptide and residues within domain 4 of another CD131 polypeptide. In one example, the residues within domain 1 of CD131 comprise residues in the region of 101-107 of SEQ ID NO: 1 and/or the residues within domain 4 of CD131 comprise residues in the region of 364-367 of SEQ ID NO: 1.

In some examples, the protein binds to an epitope comprising
a) amino acids in one CD131 polypeptide chain corresponding to one or more or all of positions 39, 101, 102, 104, 105, 106, and 107 of SEQ ID NO: 1, and
b) amino acids in another CD131 polypeptide chain corresponding to one or more or all of positions 364, 365, 366, 367, 420, and 421 of SEQ ID NO: 1.

In some examples, the protein comprises an antibody variable region comprising a V_{H} comprising three CDRs of a V_{H} comprising an amino acid sequence set forth in SEQ ID NO: 6 and a V_{L} comprising three CDRs of a V_{L} comprising an amino acid sequence set forth in SEQ ID NO: 7.

In some examples, the protein comprises an antibody variable region comprising a V_{H} comprising three CDRs of a V_{H} comprising an amino acid sequence set forth in SEQ ID NO: 6 and a V_{L} comprising three CDRs of a light chain comprising an amino acid sequence set forth in SEQ ID NO: 7.

In some examples, the protein comprises an antibody variable region comprising a V_{H} comprising three CDRs of a V_{H} comprising an amino acid sequence set forth in SEQ ID NO: 6 and a V_{L} comprising three CDRs of a V_{L} comprising an amino acid sequence set forth in SEQ ID NO: 18.

In some examples, the protein comprises
a) a V_{H} comprising a HCDR1 comprising or consisting of an amino acid sequence set forth in SEQ ID NO: 8 or a sequence having no more than one or two or three amino acid substitutions relative to SEQ ID NO: 8, a HCDR2 comprising or consisting of an amino acid sequence set forth in SEQ ID NO: 9 or a sequence having no more than one or two or three amino acid substitutions relative to SEQ ID NO: 9, and a HCDR3 comprising or consisting of an amino acid sequence set forth in SEQ ID NO: 10 or a sequence having no more than one or two or three amino acid substitutions relative to SEQ ID NO: 10; and
b) a V_{L} comprising a LCDR1 comprising or consisting of an amino acid sequence set forth in SEQ ID NO: 11 or a sequence having no more than one or two or three amino acid substitutions relative to SEQ ID NO: 11, a LCDR2 comprising or consisting of an amino acid sequence set forth in SEQ ID NO: 12 or a sequence having no more than one or two or three amino acid substitutions relative to SEQ ID NO: 12, and a LCDR3 comprising or consisting of an amino acid sequence set forth in SEQ ID NO: 13 or a sequence having no more than one or two or three amino acid substitutions relative to SEQ ID NO: 13.

In some examples, the protein comprises
a) a V_{H} comprising a HCDR1 comprising or consisting of an amino acid sequence set forth in SEQ ID NO: 8, a HCDR2 comprising or consisting of an amino acid sequence set forth in SEQ ID NO: 9, and a HCDR3 comprising or consisting of an amino acid sequence set forth in SEQ ID NO: 10; and
b) a V_{L} comprising a LCDR1 comprising or consisting of an amino acid sequence set forth in SEQ ID NO: 11, a LCDR2 comprising or consisting of an amino acid sequence set forth in SEQ ID NO: 12, and a LCDR3 comprising or consisting of an amino acid sequence set forth in SEQ ID NO: 13.

In some examples, the protein comprises a V_{H} comprising an amino acid sequence having at least 70%, at least 80%, at least 90%, or at least 95% sequence identity to SEQ ID NO: 6 and a V_{L} comprising an amino acid sequence having at least 70%, at least 80%, at least 90%, or at least 95% sequence identity to SEQ ID NO: 7.

In some examples, the protein comprises a V_{H} comprising an amino acid sequence having at least 70%, at least 80%, at least 90%, or at least 95% sequence identity to SEQ ID NO: 6 and a light chain comprising an amino acid sequence having at least 70%, at least 80%, at least 90%, or at least 95% sequence identity to SEQ ID NO: 7.

In some examples, the protein comprises a V_{H} comprising an amino acid sequence having at least 70%, at least 80%, at least 90%, or at least 95% sequence identity to SEQ ID NO: 6 and a V_{L} comprising an amino acid sequence having at least 70%, at least 80%, at least 90%, or at least 95% sequence identity to SEQ ID NO: 18.

In some examples, the protein comprises a V_{H} comprising an amino acid sequence set forth in SEQ ID NO: 6 and a V_{L} comprising an amino acid sequence set forth in SEQ ID NO: 7.

In some examples, the protein comprises a V_{H} comprising an amino acid sequence set forth in SEQ ID NO: 6 and a light chain V region comprising an amino acid sequence set forth in SEQ ID NO: 7.

In some examples, the protein comprises a V_{H} comprising an amino acid sequence set forth in SEQ ID NO: 6 and a V_{L} comprising an amino acid sequence set forth in SEQ ID NO: 18.

In some examples, the protein comprises a heavy chain comprising an amino acid sequence having at least 70%, at least 80%, at least 90%, or at least 95% sequence identity to SEQ ID NO: 14 and a light chain comprising an amino acid sequence having at least 70%, at least 80%, at least 90%, or at least 95% sequence identity to SEQ ID NO: 15.

In some examples, the protein comprises a heavy chain comprising an amino acid sequence set forth in SEQ ID NO: 14 and a light chain comprising an amino acid sequence set forth in SEQ ID NO: 15.

### KEY TO SEQUENCE LISTING

SEQ ID NO 1: amino acid sequence of Homo sapiens CD131
SEQ ID NO 2: amino acid sequence of Homo sapiens IL-3-receptor a
SEQ ID NO 3: amino acid sequence of Homo sapiens GM-CSF receptor
SEQ ID NO 4: amino acid sequence of Homo sapiens IL-5 receptor
SEQ ID NO 5: amino acid sequence of soluble Homo sapiens CD131 comprising a C-terminal 6×His tag
SEQ ID NO 6: amino acid sequence of V_{H} of 9A2-VR24.29
SEQ ID NO 7: amino acid sequence of V_{L} of 9A2-VR24.29
SEQ ID NO 8: amino acid sequence of HCDR1 of 9A2-VR24.29
SEQ ID NO 9: amino acid sequence of HCDR2 of 9A2-VR24.29
SEQ ID NO 10: amino acid sequence of HCDR3 of 9A2-VR24.29
SEQ ID NO 11: amino acid sequence of LCDR1 of 9A2-VR24.29
SEQ ID NO 12: amino acid sequence of LCDR2 of 9A2-VR24.29
SEQ ID NO 13: amino acid sequence of LCDR3 of 9A2-VR24.29
SEQ ID NO 14: amino acid sequence of heavy chain of 9A2-VR24.29
SEQ ID NO 15: amino acid sequence of light chain of 9A2-VR24.29
SEQ ID NO 16: amino acid sequence of stabilized IgG4 heavy chain constant region
SEQ ID NO 17: amino acid sequence of kappa light chain constant region
SEQ ID NO 18: an amino acid sequence of V_{L} of 9A2-VR24.29

### BRIEF DESCRIPTION OF THE DRAWINGS

FIG. 1 is a graph showing changes in ear thickness in the ACD mouse model described in Example 2. Vehicle- and DNFB-treated ear thickness was measured daily for 12 days after DNFB re-challenge. Ear swelling was determined as the difference between the ear thickness before and after each elicitation. Data: mean+S.E.M; n=5 to 11 mice with combined results from 3 independent experiments **p<0.01, Two-way ANOVA with Bonferroni's post-test for the indicated comparisons.

FIG. 2 is a graph showing changes in epidermal thickness in the ACD mouse model described in Example 2. Left panel: Epidermal thickness measurement in ears. Right panel: Difference in epidermal thickness in DNFB-treated ear/vehicle-treated ear. Data: mean; n=5 to 11 mice with combined results from 3 independent experiments ***p<0.001, Two-way ANOVA with Bonferroni's post-test for the indicated comparisons. ##p<0.01, unpaired t-test.

FIG. 3 is a graph showing changes in mast cell numbers at day 12 in the ACD mouse model described in Example 2. Left panel: Dermal mast cells number in the ear tissues at the completion of the ACD experiment were detected with toluidine blue staining. Right panel: Fold increase in mast cell number in DNFB-treated ear/vehicle-treated ear in individual mice. Data: mean+S.E.M; n=5 to 11 mice with combined results from 3 independent experiments ***p<0.001 Two-way ANOVA with Bonferroni's post-test for the indicated comparisons. ##p<0.01, unpaired t-test.

FIG. 4 is a graph showing changes in neutrophil cell numbers at day 12 in the ACD mouse model described in Example 2. Left panel: The percentage of neutrophils in the vehicle and DNFB-treated ears of different treatment groups at the completion of the ACD experiment were assessed by flow cytometry. Right panel: Fold increase in the percentage of neutrophils in DNFB-treated ear/vehicle-treated ear in individual mice.

FIG. 5 is a graph showing changes in eosinophil cell numbers at day 12 in the ACD mouse model described in Example 2. Left panel: The percentage of eosinophils in the vehicle and DNFB-treated ears of different treatment groups at the completion of the ACD experiment were assessed by flow cytometry. Right panel: Fold increase in the percentage of eosinophils in DNFB-treated ear/vehicle-treated ear in individual mice.

FIG. 6 is a graph showing changes in CD8+ T cell numbers at day 6 in the ACD mouse model described in Example 2. Left panel: The percentage of CD8+ T cells in the vehicle and DNFB-treated ears of different treatment groups at day 6 of the ACD experiment were assessed by flow cytometry. Right panel: Fold increase in the percentage of CD8+ T cells in DNFB-treated ear/vehicle-treated ear in individual mice.

FIG. 7 is a graph showing changes in neutrophil cell numbers at day 6 in the ACD mouse model described in Example 2. Left panel: The percentage of neutrophils in the vehicle and DNFB-treated ears of different treatment groups at day 6 of the ACD experiment were assessed by flow cytometry. Right panel: Fold increase in the percentage of neutrophils in DNFB-treated ear/vehicle-treated ear in individual mice.

FIG. 8 is a graph showing changes in eosinophil cell numbers at day 6 in the ACD mouse model described in Example 2. Left panel: The percentage of eosinophils in the vehicle and DNFB-treated ears of different treatment groups at day 6 of the ACD experiment were assessed by flow cytometry. Right panel: Fold increase in the percentage of eosinophils in DNFB-treated ear/vehicle-treated ear in individual mice.

FIG. 9 is a graph showing changes in mast cell numbers at day 6 in the ACD mouse model described in Example 2. Left panel: The percentage of mast cells in the vehicle and DNFB-treated ears of different treatment groups at day 6 of the ACD experiment were assessed by flow cytometry. Right panel: Fold increase in the percentage of mast cells in DNFB-treated ear/vehicle-treated ear in individual mice.

FIG. 10 is a graph showing changes in ear thickness in the AD mouse model, as described in Example 3. Ear pinna thickness was measured at 24-hour intervals over 10 days. CSL311 or isotype mAb (10 mg/kg) was injected intravenously into the tail vein of hβcTg mice at 1, 3, 5 days, as indicated by arrows.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et alt Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

For the purposes of nomenclature only and not limitation an exemplary sequence of a human CD131 (pre-CD131) is set out in NCBI Reference Sequence: NP_000386.1 and NCBI Genbank Accession Number P32927 (and set out in SEQ ID NO: 1). A sequence of a mature human CD131 lacks amino acids 1 to 16 of SEQ ID NO: 1. Positions of amino acids are often referred to herein by reference to pre-CD131. The positions in mature CD131 is readily determined by accounting for the signal sequence (amino acids 1-16 in the case of SEQ ID NO: 1). The sequence of CD131 from other species can be determined using sequences provided herein and/or in publicly available databases and/or determined using standard techniques (e.g., as described in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989)). Reference to human CD131 may be abbreviated to hCD131. Reference to soluble CD131 refers to polypeptides comprising the extracellular region of CD131, e.g., amino acids 17 to 438 of SEQ ID NO: 1.

Reference herein to CD131 includes native forms of CD131 and mutant forms thereof retaining an ability to bind to CD131 (e.g., hCD131) and induce signaling. CD131 is also known as “CSF2RB” and “cytokine receptor common subunit beta” and “β (beta) common receptor” (abbreviated as “OCR” or “c”).

A “compound”, as contemplated by the present disclosure, can take any of a variety of forms including natural compounds, chemical small molecule compounds or biological compounds or macromolecules. Exemplary compounds include an antibody or a protein comprising an antigen binding fragment of an antibody, a nucleic acid, a polypeptide, a peptide, and a small molecule.

As used herein, the term “disease” or “condition” refers to a disruption of or interference with normal function, and is not to be limited to any specific condition, disease or disorder.

As used herein, the terms “treating”, “treat” or “treatment” include administering a compound described herein to reduce, prevent, or eliminate at least one symptom of a specified disease or condition.

As used herein, the terms “preventing”, “prevent” or “prevention” include administering a compound described herein to thereby stop or hinder the development of at least one symptom of a condition, e.g., before that symptom is fully developed in the subject.

As used herein, the term “subject” shall be taken to mean any animal including humans, for example a mammal. Exemplary subjects include but are not limited to humans and non-human primates. In one example, the subject is a human.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions. In some examples, the protein is a fusion protein. As used herein, a “fusion protein” is a protein comprising at least two domains that have been joined so that they are translated as a single unit, producing a single protein.

The term “polypeptide” or “polypeptide chain” will be understood from the foregoing paragraph to mean a series of contiguous amino acids linked by peptide bonds.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

As used herein, the term “antigen binding site” shall be taken to mean a structure formed by a protein that is capable of binding or specifically binding to an antigen. The antigen binding site need not be a series of contiguous amino acids, or even amino acids in a single polypeptide chain. For example, in a Fv produced from two different polypeptide chains the antigen binding site is made up of a series of amino acids of a V_{L} and a V_{H} that interact with the antigen and that are generally, however not always in the one or more of the CDRs in each variable region. In some examples, an antigen binding site is or comprises a V_{H} or a V_{L} or a Fv. In some examples, the antigen binding site comprises one or more CDRs of an antibody.

The skilled artisan will be aware that an “antibody” is generally considered to be a protein that comprises a variable region made up of a plurality of polypeptide chains, e.g., a polypeptide comprising a V_{L} and a polypeptide comprising a V_{H}. An antibody also generally comprises constant domains, some of which can be arranged into a constant region, which includes a constant fragment or fragment crystallizable (Fc), in the case of a heavy chain. A V_{H} and a V_{L} interact to form a Fv comprising an antigen binding region that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies and chimeric antibodies.

The terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and includes amino acid sequences of complementarity determining regions (CDRs); i.e., CDR1, CDR2, and CDR3, and framework regions (FRs). Exemplary variable regions comprise three or four FRs (e.g., FR1, FR2, FR3 and optionally FR4) together with three CDRs. In the case of a protein derived from an IgNAR, the protein may lack a CDR2. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are necessary for antigen binding. Each variable region typically has three CDR regions identified as CDR1, CDR2 and CDR3. The amino acid positions assigned to CDRs and FRs can be defined according to Kabat Sequences of Proteins of Immunological Interest, National Institutes of Health, Bethesda, Md., 1987 and 1991 or other numbering systems in the performance of this disclosure, e.g., the canonical numbering system of Chothia and Lesk J. Mol Biol. 196: 901-917, 1987; Chothia et al. Nature 342, 877-883, 1989; and/or Al-Lazikani et al., J Mol Biol 273: 927-948, 1997; the IMGT numbering system of Lefranc et al., Devel. And Compar. Immunol., 27: 55-77, 2003; or the AHO numbering system of Honnegher and Plukthun J. Mol. Biol., 309: 657-670, 2001. For example, according to the numbering system of Kabat, V_{H} framework regions (FRs) and CDRs are positioned as follows: residues 1-30 (FR1), 31-35 (CDR1), 36-49 (FR2), 50-65 (CDR2), 66-94 (FR3), 95-102 (CDR3) and 103-113 (FR4). According to the numbering system of Kabat, V_{L} FRs and CDRs are positioned as follows: residues 1-23 (FR1), 24-34 (CDR1), 35-49 (FR2), 50-56 (CDR2), 57-88 (FR3), 89-97 (CDR3) and 98-107 (FR4). The present disclosure is not limited to FRs and CDRs as defined by the Kabat numbering system, but includes all numbering systems, including those discussed above. In one example, reference herein to a CDR (or a FR) is in respect of those regions according to the Kabat numbering system.

“Framework regions” (FRs) are those variable region residues other than the CDR residues.

As used herein, the term “Fv” shall be taken to mean any protein, whether comprised of multiple polypeptides or a single polypeptide, in which a V_{L} and a V_{H} associate and form a complex having an antigen binding site, i.e., capable of specifically binding to an antigen. The V_{H} and the V_{L} which form the antigen binding site can be in a single polypeptide chain or in different polypeptide chains. Furthermore, an Fv of the disclosure (as well as any protein of the disclosure) may have multiple antigen binding sites which may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of a compound or an antigen binding site thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabeled “A”), in a reaction containing labeled “A” and the protein, will reduce the amount of labeled “A” bound to the antibody.

As used herein, the term “specifically binds” or “binds specifically” shall be taken to mean that a compound of the disclosure reacts or associates more frequently, more rapidly, with greater duration and/or with greater affinity with a particular antigen or cell expressing same than it does with alternative antigens or cells. For example, a compound binds to CD131 with materially greater affinity (e.g., 20 fold or 40 fold or 60 fold or 80 fold to 100 fold or 150 fold or 200 fold) than it does to other cytokine receptors or to antigens commonly recognized by polyreactive natural antibodies (i.e., by naturally occurring antibodies known to bind a variety of antigens naturally found in humans). Generally, but not necessarily, reference to binding means specific binding, and each term shall be understood to provide explicit support for the other term.

A protein or antibody may be considered to “preferentially bind” to a polypeptide if it binds that polypeptide with a dissociation constant (K_{D}) that is less than the protein's or antibody's K_{D} for another polypeptide. In one example, a protein or antibody is considered to preferentially bind to a polypeptide if it binds the polypeptide with an affinity (i.e., K_{D}) that is at least about 20 fold or 40 fold or 60 fold or 80 fold or 100 fold or 120 fold or 140 fold or 160 fold more than the protein's or antibody's K_{D} for another polypeptide.

For the purposes of clarification and as will be apparent to the skilled artisan based on the exemplified subject matter herein, reference to “affinity” in this specification is a reference to K_{D} of a protein or antibody.

For the purposes of clarification and as will be apparent to the skilled artisan based on the description herein, reference to “a K_{D} of X nM or less” will be understood to mean that the numerical value of the K_{D} is equal to X nM or is lower in numerical value. As a skilled person would understand a lower numerical value of a K_{D} corresponds to a higher (i.e., stronger) affinity, i.e., an affinity of 2 nM is stronger than an affinity of 3 nM.

An “IC₅₀ of at least about” will be understood to mean that the IC₅₀ is equal to the recited value or greater (i.e., the numerical value recited as the IC₅₀ is lower), i.e., an IC₅₀ of 2 nM is greater than an IC₅₀ of 3 nM. Stated another way, this term could be “an IC₅₀ of X or less”, wherein X is a value recited herein.

As used herein, the term “epitope” (syn. “antigenic determinant”) shall be understood to mean a region of CD131 to which a protein comprising an antigen binding site of an antibody binds. This term is not necessarily limited to the specific residues or structure to which the protein makes contact. For example, this term includes the region spanning amino acids contacted by the protein and/or 5-10 or 2-5 or 1-3 amino acids outside of this region. In some examples, the epitope comprises a series of discontinuous amino acids that are positioned close to one another when CD131 is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

The term “competitively inhibits” shall be understood to mean that a protein of the disclosure (or an antigen binding site thereof) reduces or prevents binding of a recited antibody or protein to CD131. This may be due to the protein (or antigen binding site) and antibody binding to the same or an overlapping epitope. It will be apparent from the foregoing that the protein need not completely inhibit binding of the antibody, rather it need only reduce binding by a statistically significant amount, for example, by at least about 10% or 20% or 30% or 40% or 50% or 60% or 70% or 80% or 90% or 95%. Preferably, the protein reduces binding of the antibody by at least about 30%, more preferably by at least about 50%, more preferably, by at least about 70%, still more preferably by at least about 75%, even more preferably, by at least about 80% or 85% and even more preferably, by at least about 90%. Methods for determining competitive inhibition of binding are known in the art and/or described herein. For example, the antibody is exposed to CD131 either in the presence or absence of the protein. If less antibody binds in the presence of the protein than in the absence of the protein, the protein is considered to competitively inhibit binding of the antibody. In one example, the competitive inhibition is not due to steric hindrance.

“Overlapping” in the context of two epitopes shall be taken to mean that two epitopes share a sufficient number of amino acid residues to permit a protein (or antigen binding site thereof) that binds to one epitope to competitively inhibit the binding of a protein (or antigen binding site) that binds to the other epitope. For example, the “overlapping” epitopes share at least 1 or 2 or 3 or 4 or 5 or 6 or 7 or 8 or 9 or 20 amino acids.

### Inflammatory Skin Conditions

The methods described herein can be used to treat inflammatory skin conditions, also referred to herein and elsewhere as “inflammatory skin diseases” or “inflammatory skin disorders”. In some examples, the inflammation is non-infectious inflammation, e.g. the inflammation is not associated or caused by an infectious agent. Symptoms of an inflammatory skin disease or a skin lesion may occur at a single site (location) on a subject, or may occur at multiple sites. In some examples, one or more inflammatory skin conditions and one or more skin lesions may both occur in a subject, either at a contiguous section of skin or membrane, or at separate sites on an individual.

Inflammatory skin conditions are typically characterized by, for example, reddened, itchy, dry, rough, flaky, inflamed, and irritated skin, and the skin may also exhibit blisters, scaly plaques, etc. In some examples, the inflammatory skin condition is acute, generally resolving within days or weeks even if untreated, and the compositions and methods of the present disclosure ameliorate symptoms during disease resolution (e.g. lessen itching, redness, etc.) and/or hasten the disappearance of symptoms. Alternatively, in some examples, the skin inflammatory disease/disorder is chronic, e.g. without treatment, or even with conventional treatment, symptoms persist for weeks, months, or years, or even indefinitely. The use of the methods of the present disclosure ameliorate (provide relief from) symptoms of chronic skin inflammation while the disease persists (e.g. lessening itching, redness, cracking and flaking of skin, hastening the healing of skin lesions, etc.) and/or also partially or completely cure (cause the complete or nearly complete disappearance of) symptoms which would otherwise be present.

“Inflammatory skin condition” is intended to encompass diseases and conditions caused by exposure to specific, known or identifiable etiological agents, and also diseases/conditions whose causes are less well-defined, e.g. they are due to an immune disorder or malfunction (e.g. an autoimmune reaction), to stress, to an unidentified allergy, to a genetic predisposition, etc., and/or are due to more than one factor. “Inflammatory skin condition” is intended to encompass diseases and conditions that may have one or more other symptoms associated with it, other than just skin inflammation.

Inflammatory skin conditions (particularly chronic inflammatory skin diseases), include but are not limited to, for example hypersensitivities, autoimmune conditions (including autoimmune blistering diseases), allergic conditions and reactions, neutrophilic dermatoses, atopic conditions, autoinflammatory conditions, antibody mediated conditions and/or cell (e.g., T cell) mediated conditions.

### Atopic Dermatitis

Atopic dermatitis (AD, synonyms: neurodermitis, atopic eczema, prurigo besnier, endogenous eczema) is an inflammatory, chronically relapsing, non-contagious and intensely pruritic dermatosis characterized by epidermal inflammation, itching, dry skin (with fine scaling) and exudation (in acute lesions). It is one of the most common skin diseases with a prevalence of 2-3% in the adult population, and up to 20% in the pediatric population, with an increasing prevalence in western countries over three decades. AD usually follows a relapsing course and is associated with elevated serum immunoglobulin (IgE) levels. In this regard, one major hallmark of AD is the elevated level of total serum IgE and several therapies have been developed targeting IgE, such as omalizumab. According to current understanding, the pathophysiology of AD is the product of a complex interaction between various susceptibility genes, host environments, infectious agents, defects in skin barrier function, and immunologic responses. Most often, there is a positive family history for atopic diseases like asthma bronchiale, rhinitis allergica, conjunctivitis allergica, and/or atopic Dermatitis. These diseases are also often associated with the occurrence of AD in a subject.

The diagnosis of AD is made clinically and is based on historical features, morphology and distribution of skin lesions, and associated clinical signs. Formal sets of criteria have been developed by various groups to aid in classification. One of the most recognized sets of diagnostic criteria is the 1980 Hanifin and Rajka criteria, requiring that 3 of 4 major criteria and 3 of 23 minor criteria must be met to make the diagnosis. Predilection sites for the eczematous efflorescences of AD are the face, neck and the flexural folds of the extremities. Although predilection sites exist, the skin lesions appear without clear borders, and practically every site of the body could be involved—up to an involvement of the whole integument (erythroderma). The clinical pattern of AD also varies with age. The disease may start on the scalp, thereafter spreads to the face and extensor surfaces of the arms and legs of toddlers, sometimes showing extensive oozing and crusting. Later on, the typical preferential pattern develops with eczematous involvement of flexures, neck and hands. This is accompanied by dry skin and skin barrier dysfunction reflected by an increased trans-epidermal water loss and greater irritant skin response even involving non-lesional skin. Lichenification is a result of scratching and rubbing. Most frequently in adults this may result in the prurigo type of AD with predominant excoriated nodular lesions. Exacerbations often start as increased itch without visible skin lesions. This is then followed by erythema, papules, and infiltration in acute skin lesions. Chronic AD skin lesions have undergone tissue remodelling caused by chronic inflammation.

When compared to normal skin or uninvolved skin of patients suffering from atopic dermatitis, acute skin lesions in atopic dermatitis has a significantly greater number of IL-4, IL-5, and IL-13 mRNA-expressing cells. Dendritic cells in lesional and, to a lesser extent, in non-lesional skin of patients suffering from the disease bear IgE molecules and together with mast cells contribute to the development of a mixed lymphocytic infiltration reaction, including Th2 cells, a subset of T-cells, expressing IL-4 and IL-13 mRNA. It is thought that after an initial phase with predominantly IL-4 producing Th2 cells, a subsequent phase begins that is characterized by IFN-γ producing Th1 cells. This switch is thought to be initiated by the local production of IL-12 from infiltrating eosinophils and/or dendritic cells. Activated T-cells expressing Fas ligand have also been shown to induce keratinocyte apoptosis contributing to the spongiosis found in acute AD.

For atopic dermatitis, there is no cure available at present time. Management comprises a disease adapted treatment combining adjuvant basic therapy (emollient use) and anti-inflammatory measurements. While in very severe cases a systemic treatment with drugs (e.g. systemic glucocorticoids, ciclosporin) or UV light may be indicated for a limited period of time, topical glucocorticoids are the mainstay of the treatment also AD, with their use being well established. However, there are considerable safety concerns associated with their use, particularly when they are applied continuously and/or in patients of young age who are even more susceptible to glucocorticoid side effects, be it local cutaneous side effects (e.g. skin atrophy, telangiectasias, hypopigmentation) or systemic effects (Hypothalamic-pituitary-adrenal axis (HP A) axis suppression, growth retardation, Cushing syndrome).

### Allergic Contact Dermatitis

Allergic contact dermatitis (ACD) is regarded as a classical example of type IV hypersensitivity reaction. Unlike AD, ACD is a form of contact dermatitis, i.e., it develops as a result of xenobiotic chemicals penetrating into the skin, chemically reacting with proteins, eventually resulting in a hapten-specific T cell-mediated immune response. Thus, unlike AD, ACD is not associated with elevated IgE, making it an exception in the usage of the designation “allergic”. It is because of its well-defined localized immune response that the allergic signs and symptoms that are characteristic for ACD occur: skin redness, edema, warmth and pruritus. The diagnosis is confirmed by diagnostic patch testing, a clinically useful test that reiterates the elicitation phase of ACD. The afferent phase of the disease develops gradually over time as a result of repeated, low-grade exposures of patients to the offending chemicals.

ACD is a distinct disease entity, with well-defined mechanisms of initiation, amplification, plateau phase and disease resolution Although most environmental agents are too large to penetrate into the skin through the stratum corneum, some are of sufficiently low molecular weight to penetrate through this barrier. These molecules can be derived from naturally occurring substances, such as urushiol found in the resin of poison ivy, synthetic compounds and heavy metal ions. These compounds often are regarded as haptens, thus not being eligible to cause an allergic reaction on their own. For a sensitization reaction to occur, it is required that haptens interact with endogenous compounds (i.e. proteins) within the skin. Such a sensitization reaction has been referred to as immune recognition of ‘altered self’. That is, chemical alteration of self-molecules by xenobiotic haptens renders such self-molecules antigenic, in that this newly generated antigen (the hapten-modified self-molecule) can elicit a specific immune response.

The mechanisms of ACD involve a cascade of complex immune-mediated processes made up of two distinct phases in response to exposure to environmental chemicals, 1) the induction phase (also known as afferent or primary) and 2) the elicitation phase (also known as efferent or secondary phase).

During the induction phase of ACD, haptens applied to the skin interact with cellular proteins to form hapten-protein complexes, the antigenic moiety recognized by the immune system. These complexes are engulfed by antigen-presenting cells, such as dendritic cells, and presented in the context of MHC class II. This activates antigen-specific T-cells, which proliferate into memory T-cells. Further, NK T-cells are activated, leading to the release of cytokines including IL-2, TNF-α and IL-4. In the presence of IL-4 and antigen, B-cells also become activated and release circulating IgM.

During the elicitation phase, IgM interacts with the hapten-protein complex to induce complement activation, leading to the release of various inflammatory and chemotactic factors from mast cells and endothelial cells. Consequently antigen-specific CD8+ T-cells migrate to the site of hapten application and interact with local antigen-presenting cells, resulting in the clinical manifestations of ACD. The mixed lymphocytic infiltrate that can be observed consequently is the result of inflammatory cytokines, as well as cell-mediated cytotoxicity. Apart from the mandatory avoidance of antigens, topical glucocorticoids are the mainstay of the treatment of allergic contact dermatitis, with their use being well established. However, there are considerable safety concerns associated with their use, particularly when they are applied in patients who are susceptible to glucocorticoid side effects (e.g. due to young age), be it local cutaneous side effects (e.g. skin atrophy, telangiectasias, hypopigmentation) or systemic effects (HPA axis suppression, growth retardation, Cushing syndrome).

### Psoriasis

In some examples, the disease/condition that is treated is psoriasis, including plaque flexural, guttate, pustular, nail, photosensitive, and erythrodermic psoriasis. Psoriasis is generally recognized as an immune disorder and may be triggered by or associated with factors such as infection (e.g. strep throat or thrush), stress, injury to skin (cuts, scrapes, bug bites, severe sunburns), certain medications (including lithium, antimalarials, quinidine, indomethacin), etc. and may be comorbid with other immune conditions such as type 2 diabetes, cardiovascular disease, high blood pressure, Crohn's Disease, high cholesterol, depression, ulcerative colitis, etc. Psoriasis due to any of these causes, or any other cause or an unknown cause, may be treated by the formulations and methods described herein.

In some cases, subjects are defined as having psoriasis if they exhibit one of the following: 1) inflamed swollen skin lesions covered with silvery white scale (plaque psoriasis or psoriasis vulgaris); 2) small red dots appearing on the trunk, arms or legs (guttate psoriasis); 3) smooth inflamed lesions without scaling in the flexural surfaces of the skin (inverse psoriasis); 4) widespread reddening and exfoliation of fine scales, with or without itching and swelling (erythrodermic psoriasis); 5) blister-like lesions (pustular psoriasis); 6) elevated inflamed scalp lesions covered by silvery white scales (scalp psoriasis); 7) pitted fingernails, with or without yellowish discoloration, crumbling nails, or inflammation and detachment of the nail from the nail bed (nail psoriasis).

### Antibodies

In one example, a compound as described herein according to any example is a protein comprising an antigen binding site of an antibody. In some examples, the compound that binds to CD131 is an antibody.

Methods for generating antibodies are known in the art and/or described in Harlow and Lane (editors) Antibodies: A Laboratory Manual, Cold Spring Harbor Laboratory, (1988). Generally, in such methods CD131 or a region thereof (e.g., an extracellular domain) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, sub-cutaneously, intravenously, intradermally, intraperitoneally, or by other known route.

Monoclonal antibodies are one exemplary form of an antibody contemplated by the present disclosure. The term “monoclonal antibody” or “mAb” refers to a homogeneous antibody population capable of binding to the same antigen(s), for example, to the same epitope within the antigen. This term is not intended to be limited as regards to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

Alternatively, ABL-MYC technology (NeoClone, Madison WI 53713, USA) is used to produce cell lines secreting MAbs (e.g., as described in Largaespada et al, J. Immunol. Methods. 197: 85-95, 1996).

Antibodies can also be produced or isolated by screening a display library, e.g., a phage display library, e.g., as described in U.S. Pat. No. 6,300,064 and/or U.S. Pat. No. 5,885,793. For example, the present inventors have isolated fully human antibodies from a phage display library.

An antibody of the present disclosure may be a synthetic antibody. For example, the antibody is a chimeric antibody, a humanized antibody, a human antibody or a de-immunized antibody.

In one example, an antibody described herein is a chimeric antibody. The term “chimeric antibody” refers to antibodies in which a portion of the heavy and/or light chain is identical with or homologous to corresponding sequences in antibodies derived from a particular species (e.g., murine, such as mouse) or belonging to a particular antibody class or subclass, while the remainder of the chain(s) is identical with or homologous to corresponding sequences in antibodies derived from another species (e.g., primate, such as human) or belonging to another antibody class or subclass. Methods for producing chimeric antibodies are described in, e.g., U.S. Pat. Nos. 4,816,567; and 5,807,715.

The antibodies of the present disclosure may be humanized or human.

The term “humanized antibody” shall be understood to refer to a subclass of chimeric antibodies having an antigen binding site or variable region derived from an antibody from a non-human species and the remaining antibody structure based upon the structure and/or sequence of a human antibody. In a humanized antibody, the antigen-binding site generally comprises the complementarity determining regions (CDRs) from the non-human antibody grafted onto appropriate FRs in the variable regions of a human antibody and the remaining regions from a human antibody. Antigen binding sites may be wild-type (i.e., identical to those of the non-human antibody) or modified by one or more amino acid substitutions. In some instances, FR residues of the human antibody are replaced by corresponding non-human residues.

Methods for humanizing non-human antibodies or parts thereof (e.g., variable regions) are known in the art. Humanization can be performed following the method of U.S. Pat. No. 5,225,539, or U.S. Pat. No. 5,585,089. Other methods for humanizing an antibody are not excluded.

The term “human antibody” as used herein refers to antibodies having variable regions (e.g. V_{H}, V_{L}) and, optionally constant regions derived from or corresponding to sequences found in humans, e.g. in the human germline or somatic cells.

Exemplary human antibodies are described herein and include 9A2-VR24.29 (also, referred to as “CSL311”) described in WO 2017/088028 and BION-1 described in Sun et al. (1999) Blood 94:1943-1951 and/or proteins comprising variable regions thereof or derivatives thereof. These human antibodies provide an advantage of reduced immunogenicity in a human compared to non-human antibodies.

In one example, the antibody is a multispecific antibody. For instance, the compound that binds to CD131 may be a protein comprising an antigen binding site that binds to CD131 and a further antigen binding site that binds to a different antigen. Thus, in some examples, the antibody is a bispecific antibody.

### Antibody Binding Domain Containing Proteins

### Single-Domain Antibodies

In some examples, a compound of the disclosure is a protein that is or comprises a single-domain antibody (which is used interchangeably with the term “domain antibody” or “dAb” or “nanobody”). A single-domain antibody, is an antibody fragment consisting of a single monomeric variable antibody domain. Like a whole antibody, it is able to bind selectively to a specific antigen. With a molecular weight of only 12-15 kDa, single-domain antibodies are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain). In certain examples, a single-domain antibody is a human single-domain antibody (Domantis, Inc., Waltham, MA; see, e.g., U.S. Pat. No. 6,248,516).

In some examples, the single-domain antibody is a V_{H}H fragment. V_{H}H fragments consist of the variable domain (V_{H}) of camelid heavy-chain antibodies, described below.

In some examples, the single-domain antibody is a V_{NAR} fragment. V_{NAR} fragments consist of the variable domain (V_{H}) of heavy-chain antibodies from cartilaginous fish, described below.

### Diabodies, Triabodies, Tetrabodies

In some examples, a protein of the disclosure is or comprises a diabody, triabody, tetrabody or higher order protein complex such as those described in WO98/044001 and/or WO94/007921.

Single Chain Fv (scFv)

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favored linkers for a scFv.

### Heavy Chain Antibodies

Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR).

A general description of heavy chain antibodies from camelids and the variable regions thereof and methods for their production and/or isolation and/or use is found inter alia in the following references WO94/04678, WO97/49805 and WO 97/49805.

A general description of heavy chain antibodies from cartilaginous fish and the variable regions thereof and methods for their production and/or isolation and/or use is found inter alia in WO2005/118629.

### Other Antibodies and Antibody Fragments

The present disclosure also contemplates other antibodies and antibody fragments, such as:
(i) “key and hole” bispecific proteins as described in U.S. Pat. No. 5,731,168;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fab₃ (e.g., as described in EP19930302894).

### V-Like Proteins

An example of a compound of the disclosure is a T-cell receptor. T cell receptors have two V-domains that combine into a structure similar to the Fv module of an antibody. Novotny et al., Proc Nat Acad Sci USA 88: 8646-8650, 1991 describes how the two V-domains of the T-cell receptor (termed alpha and beta) can be fused and expressed as a single chain polypeptide and, further, how to alter surface residues to reduce the hydrophobicity directly analogous to an antibody scFv. Other publications describing production of single-chain T-cell receptors or multimeric T cell receptors comprising two V-alpha and V-beta domains include WO1999/045110 or WO2011/107595.

Other non-antibody proteins comprising antigen binding domains include proteins with V-like domains, which are generally monomeric. Examples of proteins comprising such V-like domains include CTLA-4, CD28 and ICOS. Further disclosure of proteins comprising such V-like domains is included in WO1999/045110.

### Adnectins

In one example, a compound of the disclosure is an adnectin. Adnectins are based on the tenth fibronectin type III (¹⁰F.n3) domain of human fibronectin in which the loop regions are altered to confer antigen binding. For example, three loops at one end of the β-sandwich of the ¹⁰F.n3 domain can be engineered to enable an Adnectin to specifically recognize an antigen. For further details see US20080139791 or WO2005/056764.

### Anticalins

In a further example, a compound of the disclosure is an anticalin. Anticalins are derived from lipocalins, which are a family of extracellular proteins which transport small hydrophobic molecules such as steroids, bilins, retinoids and lipids. Lipocalins have a rigid f-sheet secondary structure with a plurality of loops at the open end of the conical structure which can be engineered to bind to an antigen. Such engineered lipocalins are known as anticalins. For further description of anticalins see U.S. Pat. No. 7,250,297B1 or US20070224633.

### Affibodies

In a further example, a compound of the disclosure is an affibody. An affibody is a scaffold derived from the Z domain (antigen binding domain) of Protein A of Staphylococcus aureus which can be engineered to bind to antigen. The Z domain consists of a three-helical bundle of approximately 58 amino acids. Libraries have been generated by randomization of surface residues. For further details see EP1641818.

### Avimers

In a further example, a compound of the disclosure is an Avimer. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see WO2002/088171.

### DARPins

In a further example, a compound of the disclosure is a Designed Ankyrin Repeat Protein (DARPin). DARPins are derived from Ankyrin which is a family of proteins that mediate attachment of integral membrane proteins to the cytoskeleton. A single ankyrin repeat is a 33 residue motif consisting of two α-helices and a β-turn. They can be engineered to bind different target antigens by randomizing residues in the first α-helix and a β-turn of each repeat. Their binding interface can be increased by increasing the number of modules (a method of affinity maturation). For further details see US20040132028.

### De-Immunized Proteins

The present disclosure also contemplates a de-immunized antibody or protein. De-immunized antibodies and proteins have one or more epitopes, e.g., B cell epitopes or T cell epitopes removed (i.e., mutated) to thereby reduce the likelihood that a mammal will raise an immune response against the antibody or protein. Methods for producing de-immunized antibodies and proteins are known in the art and described, for example, in WO2000/34317, WO2004/108158 and WO2004/064724.

Methods for introducing suitable mutations and expressing and assaying the resulting protein will be apparent to the skilled artisan based on the description herein.

### Mutations to Proteins

The present disclosure also contemplates mutant forms of a protein of the disclosure. For example, such a mutant protein comprises one or more conservative amino acid substitutions compared to a sequence set forth herein. In some examples, the protein comprises 30 or fewer or 20 or fewer or 10 or fewer, e.g., 9 or 8 or 7 or 6 or 5 or 4 or 3 or 2 conservative amino acid substitutions. A “conservative amino acid substitution” is one in which the amino acid residue is replaced with an amino acid residue having a similar side chain and/or hydropathicity and/or hydrophilicity.

In one example, a mutant protein has only, or not more than, one or two or three or four or five or six conservative amino acid changes when compared to a naturally occurring protein. Details of conservative amino acid changes are provided below. As the skilled person would be aware, e.g., from the disclosure herein, such minor changes can reasonably be predicted not to alter the activity of the protein.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), 0-branched side chains (e.g., threonine, valine, isoleucine) and aromatic side chains (e.g., tyrosine, phenylalanine, tryptophan, histidine).

The present disclosure also contemplates non-conservative amino acid changes (e.g., substitutions) in a protein of the present disclosure, e.g., in a CDR, such as CDR3. In one example, the protein comprises fewer than 6 or 5 or 4 or 3 or 2 or 1 non-conservative amino acid substitutions, e.g., in a CDR3, such as in a CDR3.

The present disclosure also contemplates one or more insertions or deletions compared to a sequence set forth herein. In some examples, the protein comprises 10 or fewer, e.g., 9 or 8 or 7 or 6 or 5 or 4 or 3 or 2 insertions and/or deletions.

### Constant Regions

The present disclosure encompasses proteins and/or antibodies described herein comprising a constant region of an antibody. This includes antigen binding fragments of an antibody fused to a Fc.

Sequences of constant regions useful for producing the proteins of the present disclosure may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4. In one example, the constant region is human isotype IgG4 or a stabilized IgG4 constant region.

In one example, the Fc region of the constant region has a reduced ability to induce effector function, e.g., compared to a native or wild-type human IgG1 or IgG3 Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In one example, the Fc region is an IgG4 Fc region (i.e., from an IgG4 constant region), e.g., a human IgG4 Fc region. Sequences of suitable IgG4 Fc regions will be apparent to the skilled person and/or available in publically available databases (e.g., available from National Center for Biotechnology Information).

In one example, the constant region is a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system (Kabat et al., Sequences of Proteins of Immunological Interest Washington DC United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

Additional examples of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another example, the Fc region is a region modified to have reduced effector function, i.e., a “non-immunostimulatory Fc region”. For example, the Fc region is an IgG1 Fc region comprising a substitution at one or more positions selected from the group consisting of 268, 309, 330 and 331. In another example, the Fc region is an IgG1 Fc region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276(9):6591-604, 2001). Additional examples of non-immunostimulatory Fc regions are described, for example, in Dall'Acqua et al., J Immunol. 177: 1129-1138 2006; and/or Hezareh J Virol; 75: 12161-12168, 2001).

In another example, the Fc region is a chimeric Fc region, e.g., comprising at least one C_{H}2 domain from an IgG4 antibody and at least one C_{H}3 domain from an IgG1 antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

### Additional Modifications

The present disclosure also contemplates additional modifications to an antibody or protein of the disclosure.

For example, the antibody comprises one or more amino acid substitutions that increase the half-life of the protein. For example, the antibody comprises a Fc region comprising one or more amino acid substitutions that increase the affinity of the Fc region for the neonatal Fc receptor (FcRn). For example, the Fc region has increased affinity for FcRn at lower pH, e.g., about pH 6.0, to facilitate Fc/FcRn binding in an endosome. In one example, the Fc region has increased affinity for FcRn at about pH 6 compared to its affinity at about pH 7.4, which facilitates the re-release of Fc into blood following cellular recycling. These amino acid substitutions are useful for extending the half-life of a protein, by reducing clearance from the blood.

Exemplary amino acid substitutions include T250Q and/or M428L or T252A, T254S and T266F or M252Y, S254T and T256E or H433K and N434F according to the EU numbering system. Additional or alternative amino acid substitutions are described, for example, in US20070135620 or U.S. Pat. No. 7,083,784.

The protein may be a fusion protein. Thus, in one example, the protein additionally comprises albumin, a functional fragment or variant thereof. In one example, the albumin, functional fragment or variant thereof is serum albumin, such as human serum albumin. In one example, the albumin, functional fragment or variant thereof, comprises one or more amino acid substitutions, deletions or insertions, e.g., no more than 5 or 4 or 3 or 2 or 1 substitutions. Amino acid substitutions suitable for use in the present disclosure will be apparent to the skilled person and include naturally-occurring substitutions and engineered substitutions such as those described, for example, in WO2011/051489, WO2014/072481, WO2011/103076, WO2012/112188, WO2013/075066, WO2015/063611 and WO2014/179657.

In one example, the protein of the disclosure additionally comprises a soluble complement receptor or functional fragment or variant thereof. In one example, the protein additionally comprises a complement inhibitor.

### Protein Production

In one example, a protein described herein according to any example is produced by culturing a hybridoma under conditions sufficient to produce the protein, e.g., as described herein and/or as is known in the art.

### Recombinant Expression

In another example, a protein described herein according to any example is recombinant.

In the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. No. 4,816,567 or U.S. Pat. No. 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHOS promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

### Isolation of Proteins

Methods for isolating a protein are known in the art and/or described herein.

Where a protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO1999/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or a influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

Nucleic Acid Compounds that Bind to CD131

In one example, the compound that binds to CD131 is a nucleic acid aptamer (adaptable oligomer). Aptamers are single stranded oligonucleotides or oligonucleotide analogs that are capable of forming a secondary and/or tertiary structure that provides the ability to bind to a particular target molecule, such as a protein or a small molecule, e.g., CD131. Thus, aptamers are the oligonucleotide analogy to antibodies. In general, aptamers comprise about 15 to about 100 nucleotides, such as about 15 to about 40 nucleotides, for example about 20 to about 40 nucleotides, since oligonucleotides of a length that falls within these ranges can be prepared by conventional techniques.

An aptamer can be isolated from or identified from a library of aptamers. An aptamer library is produced, for example, by cloning random oligonucleotides into a vector (or an expression vector in the case of an RNA aptamer), wherein the random sequence is flanked by known sequences that provide the site of binding for PCR primers. An aptamer that provides the desired biological activity (e.g., binds specifically to CD131) is selected. An aptamer with increased activity is selected, for example, using SELEX (Sytematic Evolution of Ligands by EXponential enrichment). Suitable methods for producing and/or screening an aptamer library are described, for example, in Elloington and Szostak, Nature 346:818-22, 1990; U.S. Pat. No. 5,270,163; and/or U.S. Pat. No. 5,475,096.

### Assaying Activity of a Compound

### Binding to CD131

Methods for assessing binding of a candidate compound to a protein (e.g., CD131) are known in the art, e.g., as described in Scopes (In: Protein purification: principles and practice, Third Edition, Springer Verlag, 1994). Such a method generally involves labeling the protein and contacting it with immobilized compound. Following washing to remove non-specific bound protein, the amount of label and, as a consequence, bound protein is detected. Of course, the protein can be immobilized and the compound labeled. Panning-type assays can also be used. Alternatively, or additionally, surface plasmon resonance assays can be used. The level of binding can also be conveniently determined using a biosensor.

Optionally, the dissociation constant (Kd) of a compound for CD131 or an epitope thereof is determined. The “Kd” or “Kd value” for a compound that binds to CD131 is in one example measured by a radiolabeled or fluorescently-labeled CD131 binding assay. This assay equilibrates the compound with a minimal concentration of labeled CD131 in the presence of a titration series of unlabeled CD131. Following washing to remove unbound CD131, the amount of label is determined, which is indicative of the Kd of the protein.

According to another example the Kd or Kd value is measured by using surface plasmon resonance assays, e.g., using BIAcore surface plasmon resonance (BIAcore, Inc., Piscataway, NJ) with immobilized CD131 or a region thereof.

### Epitope Mapping

In another example, the epitope bound by a protein described herein is mapped. Epitope mapping methods will be apparent to the skilled artisan. For example, a series of overlapping peptides spanning the CD131 sequence or a region thereof comprising an epitope of interest, e.g., peptides comprising 10-15 amino acids are produced. The protein is then contacted to each peptide and the peptide(s) to which it binds determined. This permits determination of peptide(s) comprising the epitope to which the protein binds. If multiple non-contiguous peptides are bound by the protein, the protein may bind a conformational epitope.

Alternatively, or in addition, amino acid residues within CD131 are mutated, e.g., by alanine scanning mutagenesis, and mutations that reduce or prevent protein binding are determined. Any mutation that reduces or prevents binding of the protein is likely to be within the epitope bound by the protein.

A further method is exemplified herein, and involves binding CD131 or a region thereof to an immobilized protein of the present disclosure and digesting the resulting complex with proteases. Peptide that remains bound to the immobilized protein are then isolated and analyzed, e.g., using mass spectrometry, to determine their sequence.

A further method involves converting hydrogens in CD131 or a region thereof to deutrons and binding the resulting protein to an immobilized protein of the present disclosure. The deutrons are then converted back to hydrogen, the CD131 or region thereof isolated, digested with enzymes and analyzed, e.g., using mass spectrometry to identify those regions comprising deutrons, which would have been protected from conversion to hydrogen by the binding of a protein described herein.

Alternatively, the epitope to which the protein binds can be determined by X-ray crystallography. For example, a complex between the protein and CD131 is formed and then crystalized. The resulting crystals are then subjected to x-ray diffraction analysis to determine the atomic co-ordinates of the amino acids in the complex. The epitope comprises the amino acids in CD131 that are in contact with the protein, according to the atomic co-ordinates determined from the x-ray diffraction.

### Determining Competitive Binding

Assays for determining a protein that competitively inhibits binding of antibody 9A2-VR24.29 will be apparent to the skilled artisan. For example, 9A2-VR24.29 is conjugated to a detectable label, e.g., a fluorescent label or a radioactive label. The labeled antibody and the test protein are then mixed and contacted with CD131 or a region thereof (e.g., a polypeptide comprising SEQ ID NO: 1 or 5) or a cell expressing same. The level of labeled 9A2-VR24.29 is then determined and compared to the level determined when the labeled antibody is contacted with the CD131, region or cells in the absence of the protein. If the level of labeled 9A2-VR24.29 is reduced in the presence of the test protein compared to the absence of the protein, the protein is considered to competitively inhibit binding of 9A2-VR24.29 to CD131.

Optionally, the test protein is conjugated to different label to 9A2-VR24.29. This alternate labeling permits detection of the level of binding of the test protein to CD131 or the region thereof or the cell.

In another example, the protein is permitted to bind to CD131 or a region thereof (e.g., a polypeptide comprising SEQ ID NO: 1 or 5) or a cell expressing same prior to contacting the CD131, region or cell with 9A2-VR24.29. A reduction in the amount of bound 9A2-VR24.29 in the presence of the protein compared to in the absence of the protein indicates that the protein competitively inhibits 9A2-VR24.29 binding to CD131. A reciprocal assay can also be performed using labeled protein and first allowing 9A2-VR24.29 to bind to CD131. In this case, a reduced amount of labeled protein bound to CD131 in the presence 9A2-VR24.29 compared to in the absence of 9A2-VR24.29 indicates that the protein competitively inhibits binding of 9A2-VR24.29 to CD131.

Any of the foregoing assays can be performed with a mutant form of CD131 and/or SEQ ID NO: 1 or 5 and/or a ligand binding region of CD131 to which 9A2-VR24.29 binds, e.g., as described herein.

### Determining Neutralization

In one example, the compound that binds to CD131 reduces or prevents binding of IL-3, IL-5 and/or GM-CSF to a receptor comprising CD131 (e.g., an IL-3R, an IL-5R and/or a GM-CSF-R, respectively). These assays can be performed as a competitive binding assay using labeled IL-3/Il-5/GM-CSF and/or labeled compound. For example, cells expressing the relevant receptor is contacted with IL-3/Il-5/GM-CSF in the presence or absence of a CD131-binding compound and the amount of bound label detected. A reduction in the amount of bound label in the presence of the CD131-binding compound compared to in the absence of the compound indicates that the compound reduces or prevents binding of IL-3/Il-5/GM-CSF to a receptor comprising CD131. By testing multiple concentrations of the compound an IC₅₀ is determined, i.e., a concentration of the compound that reduces the amount of IL-3/Il-5/GM-CSF that binds to a receptor comprising CD131, or an EC₅₀ can be determined, i.e., a concentration of the protein that achieves 50% of the maximum inhibition of binding of IL-3/Il-5/GM-CSF to CD131 achieved by the compound.

In a further example, the CD131-binding compound reduces or prevents IL-3/Il-5/GM-CSF-mediated proliferation of leukemic cell line TF-1. For example, TF-1 cells are cultured without IL-3/Il-5/GM-CSF for a time sufficient for them to stop proliferating (e.g., 24-48 hours). Cells are then cultured in the presence of IL-3/Il-5/GM-CSF and various concentrations of the CD131-binding compound. Control cells are not contacted with the compound (positive control) or IL-3/Il-5/GM-CSF (negative control). Cell proliferation is then assessed using a standard technique, e.g., ³H-thymidine incorporation. A CD131-binding compound that reduces or prevents cell proliferation in the presence of IL-3 to a level less than the positive control is considered to neutralize IL-3 signaling. By testing multiple concentrations of the CD131-binding compound, an IC₅₀ is determined.

In another example, a CD131-binding compound inhibits or prevents STAT-5 activation. For example, cells (e.g., TF-1 cells) comprising a beta-lactamase reporter gene under control of the interferon regulatory factor 1 (irf1) response element in the presence of IL-3 and/or GM-CSF. Suitable cells are available from Life Technologies Corporation. Cells are also contacted with a suitable substrate (e.g., a negatively charged fluorescent beta-lactamase substrate, such as CCF2 or CCF4) and the change in signal (e.g., fluorescence) determined. A reduced change in signal in a positive control (i.e., cells contacted with IL-3 and/or GM-CSF in the absence of the compound) indicates that the compound reduces or prevents IL-3 and/or GM-CSF-induced STAT-5 signaling.

In a further example, a CD131-binding compound of the disclosure affects an immune cell. For example, the CD131-binding compound reduces or inhibits activation of isolated human neutrophils by GM-CSF as determined by reducing or inhibiting GM-CSF-induced increase in neutrophil cell size. For example, neutrophils (e.g., about 1×10⁵ cells) are cultured in the presence of the CD-131-binding protein and GM-CSF for a suitable time (e.g., about 24 hours). Cells are then fixed (e.g., with formaldehyde) and analyzed for forward scatter using flow cytometry.

In one example, the CD131-binding compound reduces or inhibits IL-3-induced IL-8 secretion by human basophils. For example, basophils (e.g., about 1×10⁵ cells) are cultured in the presence of a CD131-binding compound and IL-3 for a suitable time (e.g., 24 hours). IL-8 secretion is then assessed, e.g., using an ELISA, e.g., as is available from R&D Systems.

In a further example, the CD131-binding compound reduces or prevents IL-3-mediated survival or pDCs. For example, pDCs are cultured in the presence of a CD131-binding compound and IL-3 for a suitable time (e.g., 24 hours). Cell survival is then assessed, e.g., using a standard method, such as a ViaLight Plus Kit from Lonza.

In a further example, the CD131-binding compound reduces or prevents activation of human peripheral blood eosinophils by IL-5 as determined by assessing change in forward scatter assessed by flow cytometry. For example, eosinophils (e.g., about 1×10⁵ cells) are cultured in the presence of a CD131-binding compound and IL-5 for a suitable time (e.g., about 24 hours). Cells are then fixed (e.g., in formaldehyde) and assessed for change in forward scatter, e.g., using flow cytometry.

In a further example, a CD131-binding compound of the disclosure reduces or prevents survival of human peripheral blood eosinophils in the presence of IL-5 and/or GM-CSF and/or IL-3. For example, eosinophils (e.g., about 1×10⁴ cells) are cultured in the presence of a CD131-binding compound and IL-5 and/or GM-CSF and/or IL-3 for a suitable time (e.g., about 5 days) and cell numbers assessed using a standard method (e.g., a ViaLight Plus Kit from Lonza).

In a still further example, a CD131-binding compound of the disclosure reduces or prevents IL-3-induced TNFα release from human mast cells. For example, human cultured mast cells (e.g., ten-week old peripheral blood-derived cells) are cultured in the presence of a CD131-binding compound and IL-3. Levels of TNFα secretion are then assessed by, e.g., ELISA.

In a further example, a CD131-binding compound of the disclosure reduces or prevents IL-3-induced IL-13 release from human mast cells. For example, human cultured mast cells (e.g., ten-week old peripheral blood-derived cells) are cultured in the presence of a CD131-binding compound and IL-3. Levels of IL-13 secretion are then assessed by, e.g., ELISA.

In a further example, a CD131-binding compound of the disclosure reduces or prevents potentiation of IgE-mediated IL-8 release from human mast cells by IL-3 and/or IL-5 and/or GM-CSF. For example, human cultured mast cells (e.g., ten-week old peripheral blood-derived cells) are cultured in the presence of a CD131-binding compound and IL-3/IL-5/GM-CSF (e.g., for about 48 hours). Cells are then cultured with IgE (e.g., human myeloma IgE) for a suitable time (e.g., about 24 hours) and IL-8 secretion assessed, e.g., by ELISA.

In a further example, a CD131-binding compound reduces or prevents formation of CFU-GM by CD34+ human bone marrow cells (or cord blood cells) cultured in the presence of SCF, GM-CSF, IL-3 and IL-5. For example, CD34+ cells (e.g., about 1×10³ cells) are cultured (e.g., on methylcellulose (such as 1% methylcellulose) supplemented with fetal calf serum, bovine serum albumin, SCF, GM-CSF, IL-3 and IL-5) and in the presence of a CD131-binding compound. Cells are cultured for a suitable time (e.g., about 16 days) and the number of colonies formed subsequently enumerated.

In a further example, a CD131-binding compound reduces survival of or induces death of immune cells (e.g., eosinophils) from sputum or nasal polyp tissue from a subject suffering from an inflammatory airway disease or nasal polyposis. For example, the immune cells are cultured in the presence of IL-3 and/or IL-5 and/or GM-CSF and the protein or antibody. Cell death is then assessed using standard methods, e.g., by detecting Annexin-V expression, e.g., using fluorescence activated cell sorting).

In another example, the CD131-binding compound reduces or prevents IL-3-mediated histamine release from basophils. For example, low density leukocytes comprising basophils are incubated with IgE, IL-3 and various concentrations of the antibody or antigen binding fragment. Control cells do not comprise immunoglobulin (positive control) or IL-3 (negative control). The level of released histamine is then assessed using a standard technique, e.g., RIA. A CD131-binding compound that reduces the level of histamine release to a level less than the positive control is considered to neutralize IL-3 signaling. In one example, the level of reduction is correlated with protein concentration. An exemplary method for assessing IL-3-mediated histamine release is described, for example, in Lopez et al., J. Cell. Physiol., 145: 69, 1990.

Another assay for assessing IL-3 signaling neutralization comprises determining whether or not the CD131-binding compound reduces or prevents IL-3-mediated effects on endothelial cells. For example, human umbilical vein endothelial cells (HUVECs) are cultured in the presence of IL-3 (optionally, with IFN-γ) and various concentrations of the CD131-binding compound. The amount of secreted IL-6 is then assessed, e.g., using an enzyme linked immunosorbent assay (ELISA). Control cultures do not comprise the CD131-binding compound (positive control) or IL-3 (negative control). A CD131-binding compound that reduces or prevents IL-6 production in the presence of IL-3 to a level less than the positive control is considered to neutralize IL-3 signaling.

Other methods for assessing neutralization of GM-CSF, IL-5 or IL-3 signaling are contemplated by the present disclosure.

### Determining Effector Function

As discussed herein, some CD131-binding compounds have reduced effector function or have effector function (or enhanced effector function). Methods for assessing ADCC activity are known in the art.

In one example, the level of ADCC activity is assessed using a ⁵¹Cr release assay, an europium release assay or a ³⁵S release assay. In each of these assays, cells expressing CD131 are cultured with one or more of the recited compounds for a time and under conditions sufficient for the compound to be taken up by the cell. In the case of a ³⁵S release assay, cells expressing CD131 can be cultured with ³⁵S-labeled methionine and/or cysteine for a time sufficient for the labeled amino acids to be incorporated into newly synthesized proteins. Cells are then cultured in the presence or absence of the CD131-binding compound and in the presence of immune effector cells, e.g., peripheral blood mononuclear cells (PBMC) and/or NK cells. The amount of ⁵¹Cr, europium and/or ³⁵S in cell culture medium is then detected, and little or no change in the presence of the CD131-binding compound compared to in the absence of the CD131-binding compound indicates that the protein has reduced effector function and an increased amount compared to in the absence of the CD131-binding compound (or increased compared to in the presence of the CD131-binding compound comprising an IgG1 Fc region) indicating effector function or enhanced effector function. Exemplary publications disclosing assays for assessing the level of ADCC induced by a protein include Hellstrom, et al. Proc. Natl Acad. Sci. USA 83:7059-7063, 1986 and Bruggemann, et al., J. Exp. Med. 166:1351-1361, 1987.

Other assays for assessing the level of ADCC induced by a protein include ACTI™ nonradioactive cytotoxicity assay for flow cytometry (CellTechnology, Inc. CA, USA) or CytoTox 96® non-radioactive cytotoxicity assay (Promega, WI, USA).

C1q binding assays may also be carried out to confirm that the CD131-binding compound is able to bind C1q and may induce CDC. To assess complement activation, a CDC assay may be performed (see, for example, Gazzano-Santoro et al, J. Immunol. Methods 202: 163, 1996).

### Determining Half Life

Some proteins encompassed by the present disclosure have an improved half-life, e.g., are modified to extend their half-life compared to proteins that are unmodified. Methods for determining a protein with an improved half-life will be apparent to the skilled person.

The half-life of a protein of the disclosure can be measured by in vivo pharmacokinetic studies, e.g., according to the method described by Kim et al, Eur J of Immunol 24:542, 1994. According to this method radiolabeled protein is injected intravenously into mice and its plasma concentration is periodically measured as a function of time, for example at 3 minutes to 72 hours after the injection. Alternatively, or additionally, other species can be used, e.g. cynomolgus monkeys and humans, and/or non-radiolabeled proteins can be injected and subsequently detected using an enzyme-linked immunosorbent assay (ELISA). The clearance curve thus obtained should be biphasic, that is, an alpha phase and beta phase. For the determination of the in vivo half-life of the protein, the clearance rate in beta-phase is calculated and compared with that of the wild type or unmodified protein.

The relative affinity of binding of a protein to the neonatal Fc receptor (FcRn) can also be indicative of its relative in vivo half-life (see for example, Kim et al., Eur J Immunol., 24:2429, 1994).

### Therapeutic Efficacy

The therapeutic efficacy of a compound that binds to CD131 can be assessed by comparing the degree of severity of the disease or symptoms in subjects administered with the compound relative to subjects not administered the compound. Alternatively, or additionally, therapeutic efficacy of candidate compounds can be assessed in an animal model. Suitable assays for assessing therapeutic efficacy are described hereinabove in relation to determining neutralization by a CD131-binding compound.

In one example, the efficacy of a protein to treat a condition is assessed using an in vivo assay.

In one example, the CD131-binding compound is administered to a non-human animal (e.g., a non-human primate) and the number/level of immune cells, e.g., eosinophils, in circulation or in a tissue or other sample (e.g., skin tissue at the site of inflammation) is assessed. A CD131-binding compound that reduces the number/level of immune cells compared to prior to administration and/or in a control mammal to which the protein has not been administered is considered suitable for treating the disease or condition.

In one example, a CD131-binding compound is tested in a model of allergic contact dermatitis. In such models, a non-human mammal (e.g., a rodent, such as a mouse) is sensitized by epicutaneously applying 1-Fluoro-2,4-dinitrobenzene (DNFB) to the abdomen. The mammal is then subsequently stimulated (e.g., five days later) by epicutaneously applying DNFB to each side of its ear. The mammal is administered a CD131-binding compound and the change in ear thickness from baseline (i.e., prior to administration) and/or the level of immune cells, e.g., neutrophils, mast cells or T cells, at the site of inflammation is assessed or estimated using standard techniques. A CD131-binding compound that reduces the change in ear thickness and/or reduces the level of immune cells compared to a control mammal to which the compound has not been administered is considered suitable for treating the disease or condition.

In another example, a CD131-binding compound is tested in a model of passive cutaneous anaphylaxis, e.g., in which a non-human mammal (e.g., a rodent, such as a mouse) sensitized with anti-dinitrophenyl (DNP)-IgE and subsequently stimulated by DNP-human serum albumin (HSA) is administered a CD131-binding compound and the change in ear thickness from baseline (i.e., prior to administration) and/or the level of a cytokine, such as TNF or IL-13, is assessed or estimated using standard techniques. A CD131-binding compound that reduces the change in ear thickness and/or reduces the level of the cytokine compared to a control mammal to which the compound has not been administered is considered suitable for treating the disease or condition.

In another example, the level of an inflammatory cytokine, such as IFNα or TNFα is detected in the circulation of a mammal, e.g., using an ELISA. A CD131-binding compound that reduces the level of the cytokine compared to the level prior to administration and/or in a control mammal to which the compound has not been administered is considered suitable for treating the disease or condition.

### Compositions

In some examples, a CD131-binding compound as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, intrapolyp and intracranial injection or infusion techniques.

Methods for preparing a CD131-binding compound into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this disclosure are particularly useful for parenteral administration, such as intravenous administration or administration into a body cavity or lumen of an organ or joint. The compositions for administration will commonly comprise a solution of a CD131-binding compound dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of a CD131-binding compound of the present disclosure in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Upon formulation, a CD131-binding compound of the present disclosure will be administered in a manner compatible with the dosage formulation and in such amount as is therapeutically/prophylactically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver a CD131-binding compound of the present disclosure.

### Combination Therapies

In one example, a CD131-binding compound of the present disclosure is administered in combination with another compound useful for treating a condition described herein, either as combined or additional treatment steps or as additional components of a therapeutic formulation.

For example, the other compound is an anti-inflammatory compound. Alternatively, or additionally, the other compound is an immunosuppressant. Alternatively, or additionally, the other compound is a corticosteroid, such as prednisone and/or prednisolone. Alternatively, or additionally, the other compound is methotrexate. Alternatively, or additionally, the other compound is cyclophosphamide. Alternatively, or additionally, the other compound is mycophenolate mofetil. Alternatively, or additionally, the other compound is an anti-CD20 antibody (e.g., rituximab or ofatumumab). Alternatively, or additionally, the other compound is an anti-CD22 antibody (e.g., epratuzumab). Alternatively, or additionally, the other compound is an anti-TNF antibody (e.g., infliximab or adalimumab or golimumab) or soluble TNF receptor (e.g., etanercept). Alternatively, or additionally, the other compound is a CTLA-4 antagonist (e.g., abatacept, CTLA4-Ig). Alternatively, or additionally, the other compound is an anti-IL-6 antibody. Alternatively, or additionally, the other compound is a BLys antagonist, such as an anti-BLys antibody (e.g., belimumab).

The present disclosure also provides a method for reducing the dosage of corticosteroid required to treat a subject with an inflammatory skin condition, the method comprising co-administering a CD131-binding compound described herein and a corticosteroid, wherein the corticosteroid is administered at a lower dose than if it were administered alone or in the absence of the CD131-binding compound. The CD131-binding compound and the corticosteroid need not be administered at the same time, only in such a manner that that have an overlapping effect on the subject (e.g., are both active within the subject at the same time).

In one example, the CD131-binding compound is administered simultaneously with the other therapy. In one example, the CD131-binding compound is administered before the other therapy. In one example, the CD131-binding compound is administered after the other therapy.

In some examples, the CD131-binding compound is administered in combination with a cell. In some examples, the cell is a stem cell, such as a mesenchymal stem cell. In some examples, the CD131-binding compound signaling is administered in combination with a gene therapy.

### Dosages and Timing of Administration

Suitable dosages of a CD131-binding compound of the present disclosure will vary depending on the specific CD131-binding compound, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

In some examples, a method of the present disclosure comprises administering a prophylactically or therapeutically effective amount of a protein described herein.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present disclosure to a specific quantity, e.g., weight or amount of protein(s), rather the present disclosure encompasses any amount of the CD131-binding compound(s) sufficient to achieve the stated result in a subject.

As used herein, the term “prophylactically effective amount” shall be taken to mean a sufficient quantity of a protein to prevent or inhibit or delay the onset of one or more detectable symptoms of a clinical condition. The skilled artisan will be aware that such an amount will vary depending on, for example, the specific C131-binding protein(s) administered and/or the particular subject and/or the type or severity or level of condition and/or predisposition (genetic or otherwise) to the condition. Accordingly, this term is not to be construed to limit the present disclosure to a specific quantity, e.g., weight or amount of CD131-binding compound(s), rather the present disclosure encompasses any amount of the C131-binding protein(s) sufficient to achieve the stated result in a subject.

For in vivo administration of the CD131-binding compound described herein, normal dosage amounts may vary from about 10 ng/kg up to about 100 mg/kg of an individual's body weight or more per day. For repeated administrations over several days or longer, depending on the severity of the disease or disorder to be treated, the treatment can be sustained until a desired suppression of symptoms is achieved.

In some examples, the CD131-binding compound is administered at an initial (or loading) dose of between about 1 mg/kg to about 30 mg/kg, such as from about 1 mg/kg to about 10 mg/kg, or about 1 mg/kg or about 2 mg/kg or 5 mg/kg. The CD131-binding compound can then be administered at a lower maintenance dose of between about 0.01 mg/kg to about 2 mg/kg, such as from about 0.05 mg/kg to about 1 mg/kg, for example, from about 0.1 mg/kg to about 1 mg/kg, such as about 0.1 mg/kg or 0.5 mg/kg or 1 mg/kg. The maintenance doses may be administered every 7-30 days, such as, every 10-15 days, for example, every 10 or 11 or 12 or 13 or 14 or 15 days.

In some examples, the CD131-binding compound is administered at a dose of between about 0.01 mg/kg to about 50 mg/kg, such as between about 0.05 mg/kg to about 30 mg/kg, for example, between about 0.1 mg/kg to about 20 mg/kg, for example, between about 0.1 mg/kg to about 10 mg/kg, such as between about 0.1 mg/kg to about 2 mg/kg. For example, the CD131-binding compound is administered at a dose of between about 0.01 mg/kg to about 5 mg/kg, such as from about 0.1 mg/kg to about 2 mg/kg, such as about 0.2 mg/kg or 0.3 mg/kg or 0.5 mg/kg or 1 mg/kg or 1.5 mg/kg (e.g., without a higher loading dose or a lower maintenance dose). In some examples, numerous doses are administered, e.g., every 7-30 days, such as, every 10-22 days, for example, every 10-15 days, for example, every 10 or 11 or 12 or 13 or 14 or 15 or 16 or 17 or 18 or 19 or 20 or 21 or 22 days. For example, the CD131-binding compound is administered every 7 days or every 14 days or every 21 days.

In some examples, at the time of commencing therapy, the mammal is administered the CD131-binding compound on no more than 7 consecutive days or 6 consecutive days or 5 consecutive days or 4 consecutive days.

In the case of a mammal that is not adequately responding to treatment, multiple doses in a week may be administered. Alternatively, or in addition, increasing doses may be administered.

In another example, for mammals experiencing an adverse reaction, the initial (or loading) dose may be split over numerous days in one week or over numerous consecutive days.

Administration of a CD131-binding compound according to the methods of the present disclosure can be continuous or intermittent, depending, for example, on the recipient's physiological condition, whether the purpose of the administration is therapeutic or prophylactic, and other factors known to skilled practitioners. The administration of a CD131-binding compound may be essentially continuous over a preselected period of time or may be in a series of spaced doses, e.g., either during or after development of a condition.

### Kits

Another example of the disclosure provides kits containing compounds useful for the treatment or prevention of inflammatory skin conditions as described above.

In one example, the kit comprises (a) a container comprising a compound that b as described herein, optionally in a pharmaceutically acceptable carrier or diluent; and (b) a package insert with instructions for treating, preventing, or reducing an effect of an inflammatory skin condition in a subject.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for treating or preventing the inflammatory skin condition and may have a sterile access port (for example, the container may be an intravenous solution bag or a vial having a stopper pierceable by a hypodermic injection needle). At least one active agent in the composition is the compound that binds to CD131. The label or package insert indicates that the composition is administered to a subject eligible for treatment, e.g., one having or at risk of developing an inflammatory skin condition, with specific guidance regarding dosing amounts and intervals of compound and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The present disclosure includes the following non-limiting Examples.

### EXAMPLES

### Example 1: Materials and Methods

### Antibodies

CSL311 (ahu9A2-G4pK-VR24-29) is a humanised antibody targeting the common cytokine binding site (site 2) of the human Pc (CD131) homodimer. Heavy chain amino acid sequence is provided in SEQ ID NO: 14 and light chain amino acid sequence is provided in SEQ ID NO: 15. A human IgG4 isotype control antibody (e.g. chBM4-G4pK) was used for comparison.

### ACD (Contact Hypersensitivity) Model

Wild-type (WT) mice, βc knockout (βc^{-/-}/β_{IL-3}^{-/-}) mice and a transgenic mouse homozygous for (i) a human βc receptor transgene and (ii) knock out for both endogenous beta subunits (βc^{-/-}/β_{IL-3}^{-/-}), referred to as “hβcTg”, were used. Mouse colonies were housed at the Animal Care Facility, UniSA CRI, Building HB (Adelaide, Australia). Sixteen to twenty eight-week-old mice were used for the experiments performed in compliance with the ethical guidelines of the National Health and Medical Research Council of Australia and with approval from the University of South Australia Animal Ethics Committee (Animal ethics approval no. U21/17/).

Mice were first anaesthetised and an area of approximately a 3 cm² shaved on the posterior ventral side with an electric shaver. A pipette was used to epicutaneously apply 30 μl of 0.5% 1-Fluoro-2,4-dinitrobenzene (DNFB) solution to the abdomen of the mouse. Mice were restrained for 5-10 s to allow the solution to dry. Five days post allergen sensitisation, baseline ear pinna thickness for both ears was measured using a dial thickness gauge (model G-1A; Ozaki MFG. Co., Ltd). Immediately following measurement, 10 μl of 0.2% DNFB solution was epicutaneously applied to each side of the right ear pinna (20 μl total). Alternatively, 10 μl of 100% acetone (vehicle) was epicutaneously applied to each side of the left ear pinna (20 μl total). Twenty-four hours post elicitation, ear pinna thickness was measured at 24-hours intervals over 12 days. Changes in ear pinna thickness (A Ear swelling, mm) was calculated by the formula: (ear thickness, at each day following elicitation)−(baseline ear thickness). CSL311 or isotype mAbs (10 mg/kg) was injected intravenously into the tail vein of hβcTg mice at 1, 3, 5, days post elicitation (a total of 3 injections). Mice were killed at the end of the experiment, both ears and lymph nodes were collected for further analysis.

### Histology—Tissue Processing

DNFB-treated and vehicle-treated ear skin samples were examined histologically. Tissue samples were fixed in 10% buffered formalin, processed and embedded in paraffin and 4-μm sections cut. For tissue dewaxing and hydration, tissue sections were sequentially placed in xylene (5 min×2), 100% ethanol (4 min×2), 70% ethanol (4 min) and 50% ethanol (4 min) and then washed in deionised water.

### Toluidine Blue Staining

Dewaxed tissue sections were stained with 0.2% Toluidine Blue (pH 1.0, Sigma-Aldrich) for 90 s at room temperature and then rinsed under tap water. Sections were dehydrated with xylene and mounted with DPX mounting media. An image of the ear section was captured using a Hamamatsu nanozoomer (40× magnification, Adelaide Microscopy Facility) and exported with NDP View software (v2). Mast cells (cytoplasmic granules appear purple) per area (mm²) in the entire polyp were evaluated using Fuji Image J software (1.50e, National Institute of Health).

### Haemotoxylin and Eosin Staining

Dewaxed tissue sections were stained Haematoxylin for 3 min and then washed in running tap water for 5 min. Sections were differentiated in 1% acid-ethanol (1% HCl in 70% ethanol) for 5 min before washing in running tap water followed by staining in 1% Eosin Y solution for 10 minutes. Sections were washed in tap water for 5 minutes, then dehydrated with xylene and mounted with DPX mounting media. An image of the ear section was captured using a Hamamatsu Nanozoomer (40× magnification, Adelaide Microscopy Facility) and exported with NDP View software (v2).

### Flow Cytometric Analysis on Ear Tissues

Ear tissues were split into dorsal and ventral sections and digested with Dispase II solution (final concentration 2 U/ml) in RPMI1640 medium at 37° C. for 60 min. The dermis was then separated from the epidermis using forceps and tissues were cut into small pieces with scissors before incubation with Collagenase IV (final concentration 0.2 mg/mL) and DNase I (final concentration 0.05 mg/mL) in RPMI1640 medium at 37° C. for 60 min. Digested tissues were passed through a 70 μm nylon cell strainer (Falcon) to obtain single-cell suspensions. Cells were washed in FACS buffer (PBS with 2% FBS) prior to incubation with Live/Dead Aqua stain (1/1000 dilution, Molecular Probes) and anti-mouse CD16/CD32 antibody (eBioscience) for 15 min. For detection of mouse T cell subsets, cells were incubated for 30 min with rat anti-mouse Ly6C and Ly6G (Gr-1) PE (BD Biosciences, RB6-8C5, 1:200 dilution) and rat anti-mouse F4/80 APC (eBiosciences, BM8, 1:200 dilution) on ice for 30 min. Data was acquired as described above.

### Statistical Analysis

For normally distributed data, an unpaired t-test was used to compare different groups and two-way ANOVA with Bonferroni post-test was used for multiple comparisons. GraphPad Prism Version 5.0d was used. Asterisks above a group indicate the level of significance of that group relative to control, *p<0.05; **p<0.01; ***p<0.001.

### MC-903 Induced Atopic Dermatitis Model

Mice at 8 to 9-weeks-old with mixed genders were used in the study. Baseline ear pinna thickness for both ears was measured at day 0 using a dial thickness gauge followed by treatment with 20 μl of 100% ethanol or 20 μl of 2 nM MC-903 dissolved in 100% ethanol epicutaneously for 9 days. Ear pinna thickness was measured at 24-hour intervals over 10 days. Changes in ear pinna thickness (A Ear swelling, mm) were calculated by the formula: (ear thickness, at each day following elicitation)−(baseline ear thickness). CSL311 or isotype mAbs (10 mg/kg) were injected intravenously into the tail vein of hβcTg mice at 1, 3, 5 days (a total of 3 injections). Mice were humanely killed at the end of the experiment.

### Example 2: CSL311 Reduces Cellular Inflammation and Tissue Pathology in a hβcTg Mouse Model of Contact Dermatitis

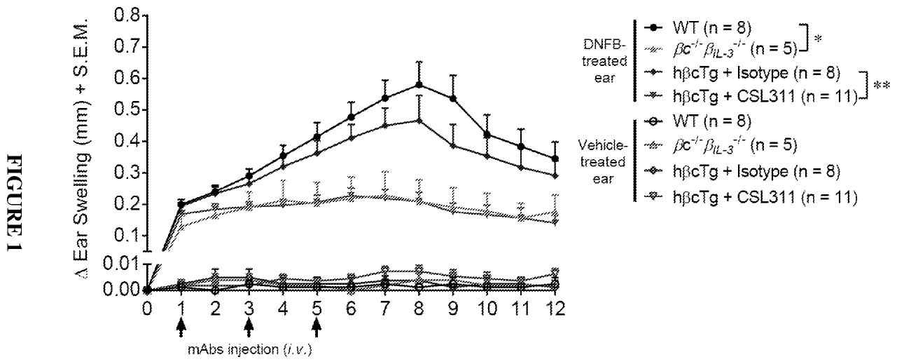

To establish the therapeutic potential of CSL311 for treatment of ACD, contact hypersensitivity reactions to an epicutaneously-applied hapten, 2,4-dinitrofluorobenzene (DNFB), were examined. WT, βc^{-/-}/β_{IL-3}^{-/-} and hβcTg mice were first sensitised by application of DNFB to the shaved abdomen and then 5 days later, DNFB was applied to the right ear and vehicle control applied to the left ear of each mouse. Changes in ear thickness (swelling) were measured each day for the proceeding 12 days and cellular inflammation, tissue visualisation and epithelial thickness measured on day 12. WT mice developed a substantial increase in ear thickness that peaked at day 7-8 and subsided thereafter. However, the magnitude of the ear swelling response was significantly reduced in the DNFB-treated ears of βc^{-/-}/β_{IL-3}^{-/-} mice that are unable to respond to any of the Pc cytokines (FIG. 1). Application of vehicle control did not induce an inflammatory response in the DNFB-sensitized mice.

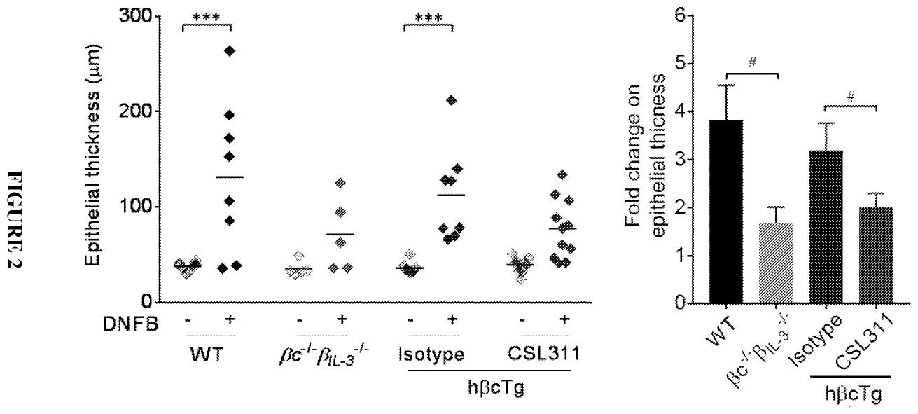

CSL311 or isotype control antibody (10 mg/kg), was intravenously injected into hβcTg mice 24 hours after the second DNFB-challenge and then every other day until day 5 (3 injections in total). Mice treated with isotype control antibody developed ear swelling responses similar to WT mice (FIG. 1). In contrast, treatment of hβcTg mice with CSL311 significantly reduced ear swelling to a level similar to that of the βc^{-/-}/β_{IL-3}^{-/-} mice (FIG. 1). There were no differences noted in vehicle-treated ears or skin at the sensitisation site, between isotype control and CSL311-treated hβcTg mice. The DNFB-treated ears of both WT mice and isotype control antibody-treated hβcTg mice were characterised by substantial dermal oedema, cellular infiltrate, hyperkeratosis and epithelial hyperplasia. These features were largely absent in DNFB-treated ears of both βc^{-/-}/β_{IL-3}^{-/-} mice and hβcTg mice treated with CSL311 (FIG. 2). These data indicate that blocking Pc cytokine signalling is an effective therapy for ACD in this model.

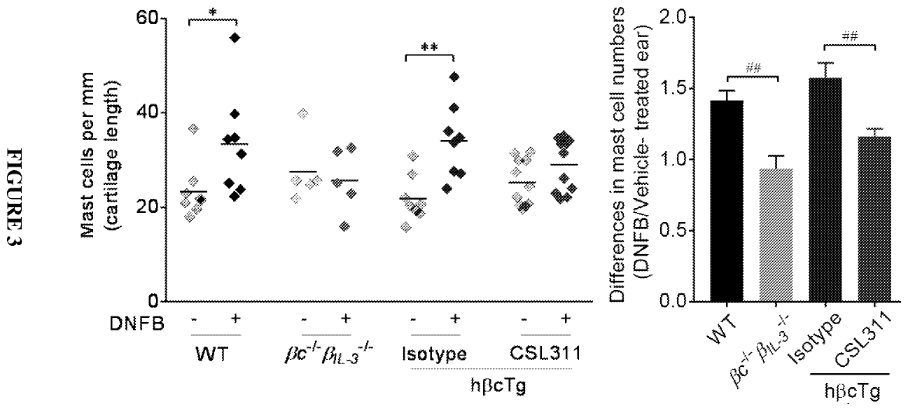

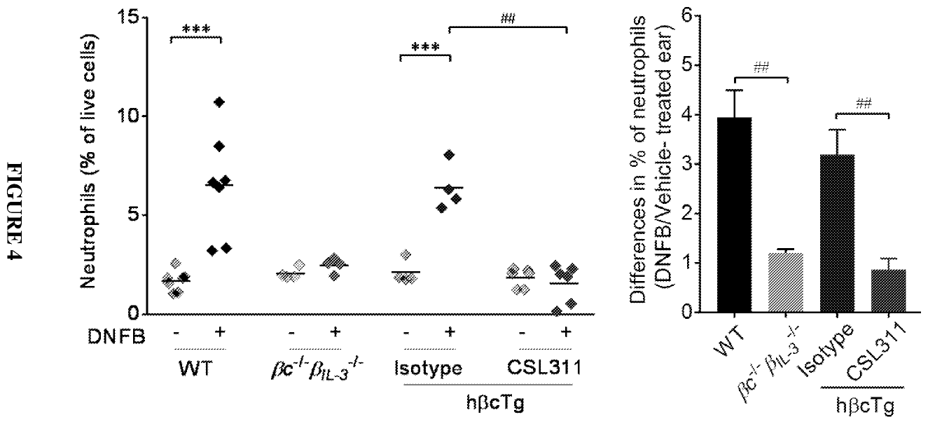

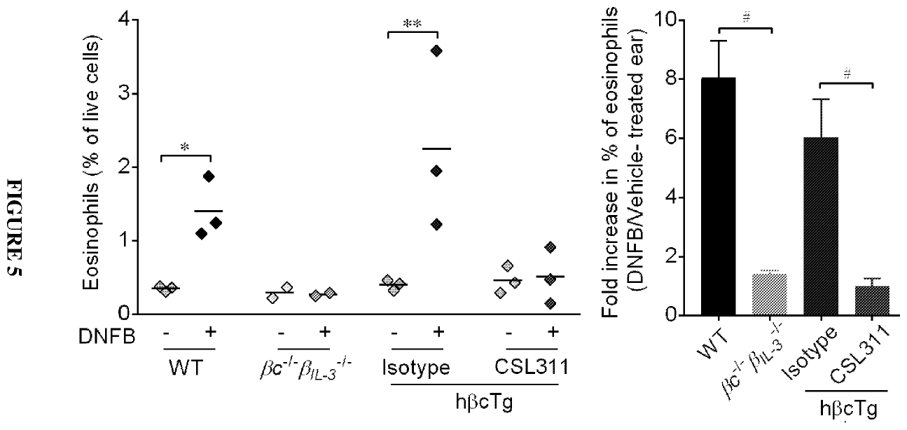

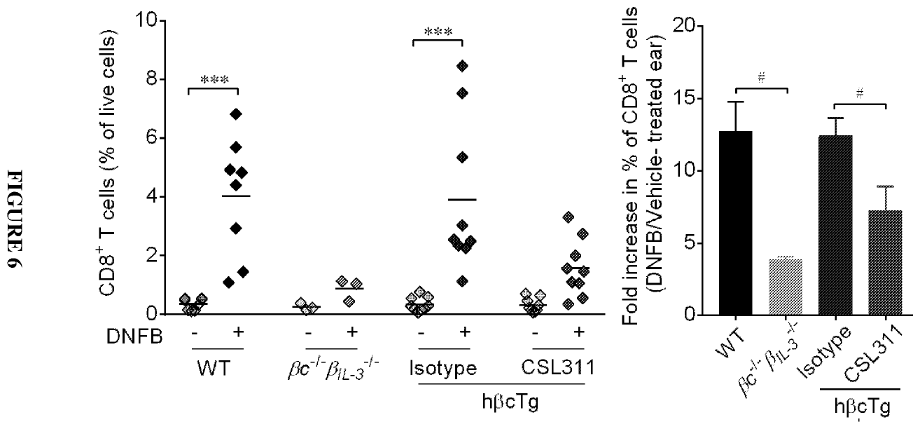

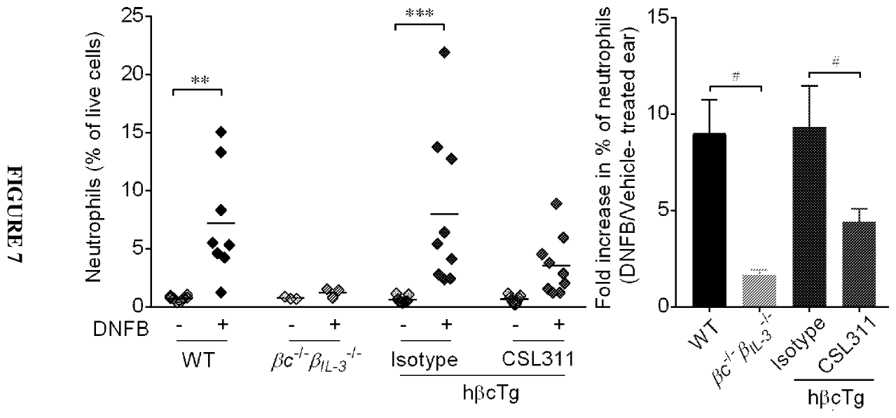

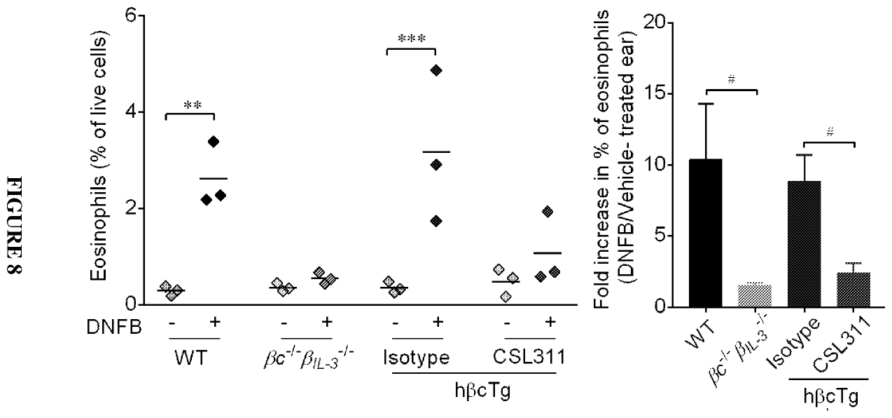

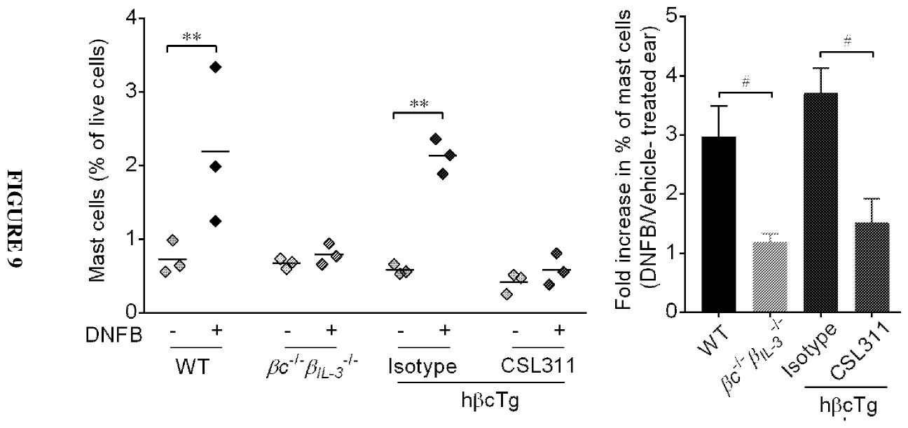

A significant increase in CD8+ T cells, mast cells, neutrophils, eosinophils and macrophages was detected in ear tissue of DNFB-treated WT mice, compared to non-DNFB-treated WT mice, at both the peak of the response (day 6) (FIGS. 6 to 9) and during the resolution phase of the response (day 12) (FIGS. 3 to 5). Absence of Pc cytokine signalling in βc^{-/-}/β_{IL-3}^{-/-} mice diminished the infiltration of all these cell types except macrophages in the DNFB-treated ears. Analysis of the ear tissues from hβcTg mice treated with the isotype control antibody revealed a significant increase in CD8+ T cells, mast cells, neutrophils and eosinophils in DNFB-treated ears at both day 6 (FIGS. 6 to 9) and day 12 (FIGS. 3 to 5) timepoints, similar to WT mice. Treatment of hβcTg mice with CSL311 abolished ear tissue accumulation of CD8+ T cells (FIG. 6), neutrophils (FIG. 7), eosinophils (FIG. 8) and mast cells (FIG. 9) at day 6 and reduced mast cells (FIG. 3), neutrophils (FIG. 4), and eosinophils (FIG. 5) at day 12 following DNFB treatment. These data therefore indicate that blockade of GM-CSF and IL-5 signalling by CSL311 limits immune cell accumulation at the site of allergen exposure and effectively reduces pathology in this hβcTg model of ACD.

The effect of CSL311 is likely mediated through blockade of either GM-CSF or IL-5 because hβcTg mice do not respond to IL-3, Moreover, these data indicate that βc cytokine-driven ear swelling is entirely dependent on GM-CSF and/or IL-5 in this ACD model because CSL311 is capable of reducing ear swelling to levels comparable to βc^{-/-}/β_{IL-3}^{-/-} mice (FIG. 1). To determine conclusively whether IL-3 contributes to contact hypersensitivity reactions in this model, changes in ear thickness in IL-3^{-/-} mice were measured using the same model described above. No differences in the ear swelling responses and mast cell infiltration between WT and IL-3^{-/-} mice were observed, indicating that GM-CSF and IL-5 are the major modulators of ACD in this model.

### Example 3: CSL311 Reduces Inflammation in a hβcTg Mouse Model of Atopic Dermatitis

The effect of CSL311 treatment in a well-characterised atopic dermatitis (AD) model was investigated. In this model (described in Example 1), daily application of vitamin D3 analogue MC903 (also known as calcipotriol) is used to induce skin inflammation. The resulting phenotype includes epidermal thickening, dermal hyperplasia, and an increased number of inflammatory cells in the skin, most notably mast cells, eosinophils, basophils and T helper type 2 cells.

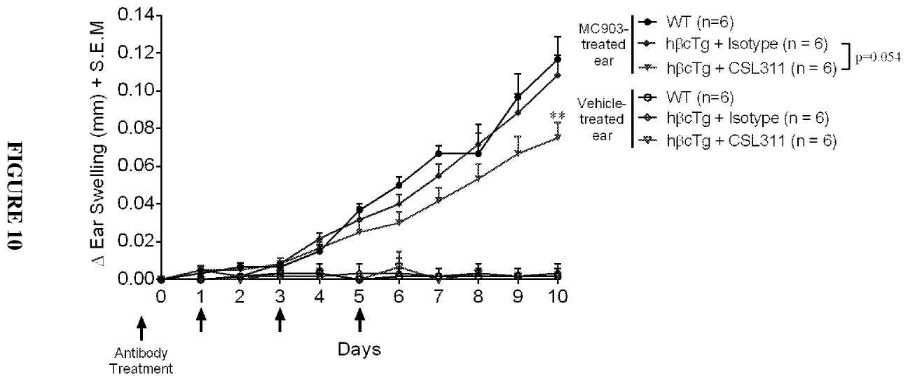

Administration of CSL311 significantly reduced ear skin thickening in hβcTg mice in this MC903-induced atopic dermatitis model (FIG. 10).

1. A method for treating an inflammatory skin condition in a subject, the method comprising administering a compound that binds to CD131 and neutralizes signaling by granulocyte-macrophage colony stimulating factor (GM-CSF) and interleukin (IL) 5 to the subject.

2. The method of claim 1, wherein the inflammatory skin condition is a hypersensitivity.

3. The method of claim 2, wherein the hypersensitivity is a type IV hypersensitivity.

4. The method of claim 1, wherein the inflammatory skin condition is a contact dermatitis or an atopic dermatitis.

5. The method of claim 4, wherein the contact dermatitis is allergic contact dermatitis.

6. The method of claim 1, wherein administration of the compound that binds to CD131 and neutralizes signaling by GM-CSF and IL-5:
a) reduces swelling at the site of inflammation, and/or
b) reduces mast cell infiltration at the site of inflammation, and/or
c) reduces neutrophil infiltration at the site of inflammation, and/or
d) reduces CD8+ T cell infiltration at the site of inflammation, and/or e) reduces eosinophil infiltration at the site of inflammation.

7. The method of claim 1, wherein the compound that binds to CD131 and neutralizes signaling by GM-CSF and IL-5 also neutralizes signaling by IL-3.

8. The method of claim 1, wherein the compound that binds to CD131 and neutralizes signaling by GM-CSF and IL-5 inhibits
a) GM-CSF-induced proliferation of TF-1 cells with an IC₅₀ of at least 100 nM; and/or
b) IL-5-induced proliferation of TF-1 cells with an IC₅₀ of at least 100 nM; and/or
c) IL-3-induced proliferation of TF-1 cells with an IC₅₀ of at least 100 nM.

9. The method of claim 1, wherein the compound that binds to CD131 and neutralizes signaling by GM-CSF and IL-5 is a protein comprising an antigen binding site that binds to CD131.

10. The method of claim 9, wherein the K_{D} of the protein for a polypeptide comprising a sequence set forth in SEQ ID NO: 5 is about 10 nM or less, when the polypeptide is immobilized on a solid surface and the K_{D} is determined by surface plasmon resonance.

11. The method of claim 1, wherein the compound that that binds to CD131 and neutralizes signaling by GM-CSF and IL-5 is a protein comprising a Fv.

12. The method of claim 11, wherein the protein comprises:
(i) a single chain Fv fragment (scFv);
(ii) a dimeric scFv (di-scFv);
(iii) a diabody;
(iv) a triabody;
(v) a tetrabody;
(vi) a Fab;
(vii) a F(ab′)₂;
(viii) a Fv;
(ix) one of (i) to (viii) linked to a constant region of an antibody, Fc or a heavy chain constant domain (C_{H}) 2 and/or C_{H}3;
(x) one of (i) to (viii) linked to albumin or a functional fragment or variants thereof or a protein that binds to albumin; or
(xi) an antibody.

13. The method of claim 9, wherein the protein is a single domain antibody (sdAb).

14. The method of claim 9, wherein the protein comprises an antibody variable region that binds to CD131 and competitively inhibits the binding of antibody 9A2-VR24.29 comprising a V_{H} comprising a sequence set forth in SEQ ID NO: 6 and a V_{L} comprising a sequence set forth in SEQ ID NO: 18 to CD131.

15. The method of claim 9, wherein the antigen binding site binds:
(a) to an epitope within Site 2 of CD131; and/or
(b) to an epitope formed upon dimerization of two CD131 polypeptides; and/or
(c) to residues within domain 1 of a CD131 polypeptide and residues within domain 4 of another CD131 polypeptide.

16. (canceled)

17. (canceled)

18. The method of claim 15, wherein the residues within domain 1 of CD131 comprise residues in the region of 101-107 of SEQ ID NO: 1 and/or the residues within domain 4 of CD131 comprise residues in the region of 364-367 of SEQ ID NO: 1.

19. The method of claim 9, wherein the protein comprises;
(a) an antibody variable region comprising a V_{H} comprising three CDRs of a V_{H} comprising an amino acid sequence set forth in SEQ ID NO: 6 and a V_{L} comprising three CDRs of a V_{L} comprising an amino acid sequence set forth in SEQ ID NO: 18; and/or
(b) a V_{H} comprising an amino acid sequence set forth in SEQ ID NO: 6 and a V_{L} comprising an amino acid sequence set forth in SEQ ID NO: 18; and/or
(c) a heavy chain comprising an amino acid sequence set forth in SEQ ID NO: 14 and a light chain comprising an amino acid sequence set forth in SEQ ID NO: 15.

20. (canceled)

21. (canceled)


SEQUENCE LISTING (18 sequences)

SEQ ID NO: 1 - protein, 897 aa, Homo sapiens
MVLAQGLLSMALLALCWERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESVLPMWVLALIVIFLTIAVLLALRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 2 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 3 - protein, 836 aa, Homo sapiens
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 4 - protein, 420 aa, Homo sapiens
MIIVAHVLLILLGATEILQADLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICHLWIKLFPPIPAPKSNIKDLFVTTNYEKAGSSETEIEVICYIEKPGVETLEDSVF

SEQ ID NO: 5 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 6 - protein, 117 aa, Artificial Sequence
  Synthetic antibody sequence
EVQLLESGGGLVQPGGSLRLSCAASGFTFPWYRVHWVRQAPGKGLEWVSSIRSSGGFPYYNYKVKGRFTISRDNSKNTLYLQMNSLRAEDTAVYYCARFYDSFFDIWGQGTMVTVSS

SEQ ID NO: 7 - protein, 112 aa, Artificial Sequence
  Synthetic antibody sequence
DIQMTQSPSSVSASVGDRVTITCRASQGISSWLAWYQQKPGKAPKLLIYAASSLQSGVPSRFSGSGSGTDFTLTISSLQPEDFATYYCQQANSFPITFGQGTRLEIKRTVAA

SEQ ID NO: 8 - protein, 10 aa, Artificial Sequence
  Synthetic antibody sequence
GFTFPWYRVH

SEQ ID NO: 9 - protein, 17 aa, Artificial Sequence
  Synthetic antibody sequence
SIRSSGGFPYYNYKVKG

SEQ ID NO: 10 - protein, 8 aa, Artificial Sequence
  Synthetic antibody sequence
FYDSFFDI

SEQ ID NO: 11 - protein, 11 aa, Artificial Sequence
  Synthetic antibody sequence
RASQGISSWLA

SEQ ID NO: 12 - protein, 7 aa, Artificial Sequence
  Synthetic antibody sequence
AASSLQS

SEQ ID NO: 13 - protein, 9 aa, Artificial Sequence
  Synthetic antibody sequence
QQANSFPIT

SEQ ID NO: 14 - protein, 444 aa, Artificial Sequence
  Synthetic antibody sequence
EVQLLESGGGLVQPGGSLRLSCAASGFTFPWYRVHWVRQAPGKGLEWVSSIRSSGGFPYYNYKVKGRFTISRDNSKNTLYLQMNSLRAEDTAVYYCARFYDSFFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 214 aa, Artificial Sequence
  Synthetic antibody sequence
DIQMTQSPSSVSASVGDRVTITCRASQGISSWLAWYQQKPGKAPKLLIYAASSLQSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 327 aa, Artificial Sequence
  Synthetic antibody sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 104 aa, Artificial Sequence
  Synthetic antibody sequence
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 108 aa, Artificial Sequence
  Synthetic antibody sequence
DIQMTQSPSSVSASVGDRVTITCRASQGISSWLAWYQQKPGKAPKLLIYAASSLQSGVPSRFSGSGSGTDFTLTISSLQPEDFATYYCQQANSFPITFGQGTRLEIKR